(19) United States
(12) Patent Application Publication
(10) Pub. No.: US 2013/0109985 A1
(43) Pub. Date: May 2, 2013

(54) FAULT-TOLERANT SENSING IN AN IMPLANTABLE MEDICAL DEVICE

(71) Applicants: Jeffrey M. Gillberg, Coon Rapids, MN (US); Scott A. Hareland, Lino Lakes, MN (US); Leonard P. Radtke, St. Michael, MN (US); David G. Schaenzer, Minneapolis, MN (US); John D. Wahlstrand, Shoreview, MN (US)

(72) Inventors: Jeffrey M. Gillberg, Coon Rapids, MN (US); Scott A. Hareland, Lino Lakes, MN (US); Leonard P. Radtke, St. Michael, MN (US); David G. Schaenzer, Minneapolis, MN (US); John D. Wahlstrand, Shoreview, MN (US)

(73) Assignee: Medtronic, Inc., Minneapolis, MN (US)

(21) Appl. No.: 13/285,872

(22) Filed: Oct. 31, 2011

(51) Int. Cl. A61B 5/0402

(52) U.S. Cl. 600/509

(57) ABSTRACT

A system includes a memory and a processing module. The memory includes a primary sensing vector and N alternate sensing vectors. The processing module determines a ranking value for each of the N alternate sensing vectors. Each ranking value is indicative of the integrity of a cardiac electrical signal acquired via the corresponding alternate sensing vector. The processing module senses cardiac events using the primary sensing vector, detects a reduction in the integrity of a cardiac electrical signal acquired via the primary sensing vector, and selects one of the N alternate sensing vectors in response to detecting a reduction in the integrity of the cardiac electrical signal acquired via the primary sensing vector. The selection is based on the ranking value associated with the one of the N alternate sensing vectors. The processing module then senses cardiac events using the selected one of the N alternate sensing vectors.

### TECHNICAL FIELD

The disclosure relates to techniques for providing fault tolerance in an implantable medical device, and more particularly, to techniques for providing tolerance to faults in a sensing pathway of an implantable medical device.

### BACKGROUND

Implantable medical devices (IMDs), such as implantable cardioverter-defibrillators and implantable pacemakers, may sense cardiac electrical activity using one or more sensing vectors. For example, IMDs may sense ventricular events (e.g., ventricular contractions) using a ventricular sensing vector. IMDs may implement a variety of different algorithms in order to detect arrhythmias based on the ventricular events sensed using the ventricular sensing vector. In some examples, IMDs may implement rate-based detection and analysis algorithms in order to detect and analyze bradyarrhythmias and/or tachyarrhythmias. An IMD that implements a rate-based detection algorithm may monitor the length of intervals between sensed ventricular events, and detect a tachyarrhythmia when a predetermined number of those intervals are shorter than a programmed time interval. In some examples, IMDs may perform further analysis of arrhythmias using rate, pattern, and signal morphology information. For example, IMDs may characterize arrhythmias based on the range of values in which the intervals fall, the stability of the intervals, the average or median values of the intervals, the onset of intervals, and the morphology of electrogram waveforms.

IMDs may provide a variety of therapies in response to a detected arrhythmia. In some examples, an IMD may provide anti-tachyarrhythmia pacing (ATP) in order to correct a detected tachycardia. In other examples, an IMD may deliver high-energy therapy (e.g., cardioversion or defibrillation) to a patient when a potentially life-threatening arrhythmia is detected, such as ventricular tachycardia or ventricular fibrillation. In still other examples, an IMD may detect bradyarrhythmias or low heart rate and provide pacing at some minimum rate or rate determined by rate responsive sensors to ensure adequate cardiac output.

### SUMMARY

The IMD of the present disclosure may sense ventricular events and detect arrhythmias using a primary sensing vector. In some scenarios, the sensing integrity of the primary sensing vector may be compromised and the integrity of the cardiac electrical signals acquired via the primary sensing vector may degrade. As a result of the reduction in integrity of the cardiac electrical signals acquired via the compromised primary sensing vector, the IMD may inappropriately sense ventricular events and inappropriately detect tachyarrhythmias, provide inappropriate tachyarrhythmia therapy, and withhold needed bradyarrhythmia therapy in some examples.

The IMD of the present disclosure may periodically determine whether the primary sensing vector is compromised using one or more sensing integrity measurements. For example, the IMD may determine when the primary sensing vector is compromised based on a detection of a fault associated with the primary sensing vector, detected noise associated with the primary sensing vector, or a detected decrease in the amplitude of signals acquired via the primary sensing vector. In response to determining that the primary sensing vector is compromised, the IMD may select one of a plurality of alternate sensing vectors from memory and set the selected alternate sensing vector as the new primary sensing vector. Subsequently, the IMD may use the newly selected primary sensing vector to sense ventricular events and to detect arrhythmias. In some examples, the switch from a compromised primary sensing vector to one of the alternate sensing vectors may bypass the source of the reduction in signal integrity associated with the primary sensing vector. In a similar manner, the IMD may also select one of a plurality of alternate pacing vectors from memory to ensure proper bradyarrhythmia therapy. In some examples, the sensing and pacing vectors may both be changed to the same new alternate vector. In other examples, the pacing and sensing vectors may be switched independently from each other.

Each of the alternate sensing vectors from which the IMD may select may be associated with a ranking value that indicates the integrity of a signal that may be acquired using that alternate sensing vector. After determining that the sensing integrity of the primary sensing vector is compromised, the IMD of the present disclosure may identify an alternate sensing vector having a high sensing integrity by identifying a ranking value that indicates a high sensing integrity. Subsequently, the IMD may select the alternate sensing vector that corresponds to a ranking value having the high sensing integrity.

The IMD of the present disclosure may update the ranking values during operation. The IMD may perform a variety of different types of sensing integrity measurements in order to update the ranking values. Example integrity measurements that may be performed by the IMD to determine the sensing integrity of the alternate sensing vectors may include, but are not limited to, impedance measurements, noise measurements, capture threshold measurements, and signal amplitude measurements. By updating the ranking values during operation, the IMD may ensure that the ranking values associated with the alternate sensing vectors reflect a current sensing integrity of the alternate sensing vectors. Accordingly, the IMD may use the ranking values to reliably select which alternate sensing vector to use to replace the primary sensing vector in the event that the IMD detects that the primary sensing vector is compromised. The periodic updates to the ranking values may help to ensure that the alternate sensing vector selected by the IMD is reliable at the time of switching. In other words, the periodic updates to the ranking values may prevent the IMD from switching from a compromised primary sensing vector to an alternate sensing vector that may also have a sensing integrity issue.

In one example according to the present disclosure, a system comprises a memory and a processing module. The memory comprises a primary sensing vector and N alternate sensing vectors. N is an integer that is greater than 1. The processing module is configured to determine a ranking value for each of the N alternate sensing vectors. Each ranking value is indicative of the integrity of a cardiac electrical signal acquired via the corresponding alternate sensing vector. The processing module is further configured to sense cardiac events using the primary sensing vector, detect a reduction in the integrity of a cardiac electrical signal acquired via the primary sensing vector, and select one of the N alternate sensing vectors in response to detecting a reduction in the integrity of the cardiac electrical signal acquired via the primary sensing vector. The selection is based on the ranking value associated with the one of the N alternate sensing vectors. Additionally, the processing module is configured to sense cardiac events using the selected one of the N alternate sensing vectors.

In another example according to the present disclosure, a method comprises storing a primary sensing vector and N alternate sensing vectors in a memory. N is an integer that is greater than 1. The method further comprises determining a ranking value for each of the N alternate sensing vectors. Each ranking value is indicative of the integrity of a cardiac electrical signal acquired via the corresponding alternate sensing vector. The method further comprises sensing cardiac events using the primary sensing vector, detecting a reduction in the integrity of a cardiac electrical signal acquired via the primary sensing vector, and selecting one of the N alternate sensing vectors in response to detecting a reduction in the integrity of the cardiac electrical signal acquired via the primary sensing vector. The selection is based on the ranking value associated with the one of the N alternate sensing vectors. Additionally, the method comprises sensing cardiac events using the selected one of the N alternate sensing vectors.

In another example according to the present disclosure, a method comprises sensing a plurality of ventricular events using a first ventricular sensing vector, detecting a plurality of arrhythmias based on analysis of the plurality of sensed ventricular events, and determining whether to withhold therapy for each of the plurality of detected arrhythmias. The method further comprises determining a number of times that therapy was withheld for the plurality of detected arrhythmias and determining when to switch from the first ventricular sensing vector to a second ventricular sensing vector based on the number of times therapy was withheld.

In another example according to the present disclosure, a system comprises a memory and a processing module. The memory comprises a first ventricular sensing vector and a second ventricular sensing vector. The processing module is configured to sense a plurality of ventricular events using the first ventricular sensing vector, detect a plurality of arrhythmias based on analysis of the plurality of sensed ventricular events, and determine whether to withhold therapy for each of the plurality of detected arrhythmias. The processing module is further configured to determine a number of times that therapy was withheld for the plurality of detected arrhythmias and determine when to switch from the first ventricular sensing vector to the second ventricular sensing vector based on the number of times therapy was withheld.

In another example according to the present disclosure, a system comprises a memory and a processing module. The memory comprises a primary pacing vector and N alternate pacing vectors. N is an integer that is greater than 1. The processing module is configured to determine a ranking value for each of the N alternate pacing vectors. Each ranking value is indicative of the integrity of the corresponding alternate pacing vector. The processing module is further configured to pace one of the atria and the ventricles using the primary pacing vector, detect a reduction in the integrity of the primary pacing vector, and select one of the N alternate pacing vectors in response to detecting a reduction in the integrity of the primary pacing vector. The selection is based on the ranking value associated with the one of the N alternate pacing vectors. Additionally, the processing module is configured to pace the one of the atria and the ventricles using the selected one of the N alternate pacing vectors.

The details of one or more examples are set forth in the accompanying drawings and the description below. Other features, objects, and advantages will be apparent from the description and drawings, and from the claims.

### BRIEF DESCRIPTION OF THE DRAWINGS

FIG. 1 shows an example system including an implantable medical device (IMD) that may be used to diagnose conditions of and provide therapy to a heart of a patient.

FIG. 2 shows a detailed view of the IMD of FIG. 1.

FIG. 3 shows a functional block diagram of an example IMD.

FIGS. 4A-4B show example sensing vector tables included in a memory of an IMD.

FIG. 5 is a flowchart that illustrates an example method for reconfiguring a primary sensing vector in response to a determination that the sensing integrity of the primary sensing vector is compromised.

FIG. 6 is a flowchart that illustrates an example method for reconfiguring a primary sensing vector in response to a determination that the sensing integrity of the primary sensing vector is compromised based on a number of withheld therapies.

FIGS. 7A-7C are functional block diagrams that illustrate detection of a mechanical fault in a primary sensing vector and subsequent reconfiguration of the primary sensing vector.

FIG. 8 is a flowchart that illustrates an example method for updating alternate sensing vectors of a sensing vector table.

FIGS. 9A-9B illustrate updating of alternate sensing vectors in a sensing vector table and subsequent selection of a new primary sensing vector from the updated sensing vector table.

### DETAILED DESCRIPTION

An IMD of the present disclosure may sense cardiac electrical activity using one or more sensing vectors. A sensing vector may include a pair of electrodes that may be used by the IMD to sense cardiac electrical activity. A ventricular sensing vector may describe a pair of electrodes used to sense ventricular activity (e.g., the QRS complex). An atrial sensing vector may describe a pair of electrodes used to sense atrial activity (e.g., P waves). Although an IMD may sense ventricular and atrial activity using ventricular and atrial sensing vectors, respectively, the phrase “sensing vector” as used hereinafter may describe a ventricular sensing vector. Accordingly, a “sensing vector” as described hereinafter may be used for ventricular sensing, i.e., sensing of a ventricular depolarization events (i.e., ventricular events).

Although the present disclosure describes sensing of ventricular events using a ventricular sensing vector and subsequent reconfiguration of the ventricular sensing vector in response to a determination that the sensing integrity of the ventricular sensing vector is compromised, reconfiguration of other vectors according the present disclosure is contemplated. For example, the systems and methods of the present disclosure may be applicable to using an atrial sensing vector to sense atrial events and subsequent reconfiguration of the atrial sensing vector in response to a determination that the sensing integrity of the atrial sensing vector is compromised. Additionally, or alternatively, the systems and methods of the present disclosure may be applicable to reconfiguring pacing vectors of the IMD that may be used to pace the atria or ventricles.

Example electrodes of a ventricular sensing vector may include, but are not limited to, electrodes on a right ventricular lead (e.g., RVtip, RVring, right ventricular coil HVB, and SVC coil (HVX)), electrodes on a left ventricular lead (e.g., LVtip and one or more LVring electrodes), epicardial or patch electrodes, and the can electrode HVA. Ventricular sensing vectors may include electrodes on the same lead (e.g., RVtip-RVring, RVtip-HVB, and LVtip-LVring), electrodes on different leads (e.g., RVtip-LVring), or an electrode on a lead in addition to the can electrode (e.g., RVtip-HVA).

The IMD of the present disclosure may include a memory that stores a plurality of different ventricular sensing vectors, such as RVtip-RVring, RVtip-HVB, LVtip-LVring, RVtip-LVring, and RVtip-HVA. The IMD may use one of the plurality of ventricular sensing vectors as a primary sensing vector. The primary sensing vector may refer to the electrode combination that the IMD uses to sense ventricular events (e.g., ventricular depolarizations). The IMD may detect arrhythmias based on the ventricular events sensed using the primary sensing vector. For example, the IMD may detect tachyarrhythmias based on a rate of ventricular sensed events. In some examples, a clinician may select the primary sensing vector from a plurality of different sensing vectors prior to, or upon, implantation of the IMD in the patient. The clinician may set the primary sensing vector as the sensing vector that the clinician expects to be the most reliable sensing vector for sensing ventricular events. In some examples, as described herein, the clinician may set the primary sensing vector as the RVtip-RVring sensing vector. In other examples, the primary sensing vector may be a factory default setting used by the IMD based, for example, on a common electrode vector used.

The primary sensing vector may initially be selected (e.g., by a clinician, or as a default) based on the assumption that the primary sensing vector does not include potential sensing integrity issues. The integrity of a sensing vector may generally refer to quality (i.e., integrity) of the cardiac electrical signal that may be obtained by the IMD using the sensing vector. Generally, a high sensing integrity associated with a sensing vector may indicate that the sensing vector may reliably acquire a high quality cardiac electrical signal that the IMD may confidently depend upon in order to sense ventricular events and detect arrhythmic episodes. A low sensing integrity associated with a sensing vector may indicate that the cardiac electrical signal acquired via the sensing vector may be deficient in one or more ways such that electrical signals sensed via the sensing vector may not be as reliable as in the case when the sensing vector has a high sensing integrity. In other words, the IMD may not confidently rely on cardiac electrical signals received via a sensing vector that has a low sensing integrity. In some examples, the IMD may have a tendency to inappropriately sense ventricular events and inappropriately detect arrhythmias or other heart activity when using a sensing vector having a low sensing integrity.

The sensing integrity of a sensing vector may be affected by a variety of different factors. In one example, a reduction in sensing integrity may be caused by a variety of different faults or environmental conditions. Such sensing integrity issues, when present in the primary sensing vector, may cause errors in sensing of ventricular events and may also cause inappropriate detection of arrhythmias. A reduction in sensing integrity may be caused by mechanical faults (e.g., broken conductors in leads, incomplete connection of a lead to a connector on the IMD, lead-to-lead interaction), noise, such as non-physiological noise (e.g., electromagnetic interference (EMI)) and physiological noise (e.g., associated within movement of tissue proximate to electrodes due to patient activity, such as movement of muscles proximate to the HVA electrode on the can, or lead-to-physiology interaction such as RVC-to-tricuspid interaction). Additionally, a reduction in sensing integrity may be caused by changes in the electrode-tissue interface, such as development of heart tissue damage and fibrotic encapsulation of electrodes. The reduction in sensing integrity may be manifested in a variety of ways, e.g., short intervals, intermittent or consistently high or low lead impedances, oversensing resulting in inappropriate detection of non-sustained tachyarrhythmias, loss or variability of signal amplitude, increase in high frequency noise on a sensing signal, or other oversensing such as T-wave oversensing, etc.

Mechanical faults in the IMD may cause a reduction in the integrity of a sensing vector. In one example, mechanical faults may include a fracture in a conductor included in a lead of the IMD (i.e., lead fractures). A lead fracture may present a high impedance in the sensing path which may reduce the sensing integrity of a sensing vector that uses that sensing path. Such a high impedance may be intermittent or continuous. Similarly, a disconnection of a lead, or inadequate mechanical stabilization of a lead in the connector block of the IMD may cause a high impedance in the sensing path that may reduce sensing integrity. In some examples, intermittent mechanical faults in the lead path or in the connector block may cause the IMD to falsely detect ventricular events and inappropriately interpret the events as an arrhythmia. Accordingly, a mechanical fault, such as a lead fracture or a disconnection of the lead from the connector block, may reduce the sensing integrity of a sensing vector associated with the mechanical fault.

The mechanical faults described above may be detected using a variety of different detection techniques. For example, the IMD may detect lead fractures and/or disconnections of a lead using a lead impedance test. Additionally, or alternatively, the IMD may detect lead fractures and/or disconnections of a lead based on a number/frequency of short intervals, observations of non-sustained tachyarrhythmias or non-physiologic morphologies, comparisons between one or more other sensing vectors, independent sensors that detect cardiac activity such as heart sounds, cardiac accelerometers and/or hemodynamic sensors, or using circuits that are configured to detect different faults in the leads, connectors, or active IMD electronics.

Sensing vectors that may be prone to picking up noise, such as EMI, may have a reduced sensing integrity as compared to sensing vectors that are less prone to picking up environmental noise. In some examples, electrical noise may be induced, e.g., by EMI, in the conductors included in the primary sensing vector. Noise induced in the primary sensing vector may cause the IMD to inappropriately sense ventricular events and inappropriately detect arrhythmias. The IMD may detect EMI and other environmental noise using various digital signal processing algorithms, e.g., to ascertain non-physiologic frequencies, variability, amplitudes, or waveform morphologies. Some detection techniques may include monitoring alternative vectors and/or sensor data.

The IMD of the present disclosure may quantify the sensing integrity of the primary sensing vector using a variety of different techniques described herein, e.g., at least one of impedance measurements, noise measurements, and amplitude measurements. The IMD may determine whether the integrity of the primary sensing vector has been compromised based on the quantified sensing integrity of the primary sensing vector. The IMD may determine that the sensing integrity of the primary sensing vector has been compromised when the IMD has gathered enough quantitative evidence to make the decision that the sensing integrity of the primary sensing vector has been reduced to such a level that using the primary sensing vector to sense ventricular events may be unreliable.

The primary sensing vector may be stored in the memory along with a plurality of alternate sensing vectors. Typically, the IMD may sense ventricular events and detect arrhythmias using the primary sensing vector. However, when the IMD determines that the sensing integrity of the primary sensing vector is compromised (e.g. based on a detected fault or detected noise), the IMD may select one of the alternate sensing vectors from memory and set the selected alternate sensing vector as the new primary sensing vector. Subsequently, the IMD may use the newly selected primary sensing vector to sense ventricular events and to detect arrhythmias. Switching a current primary sensing vector to one of the alternative sensing vectors when the current primary sensing vector has been compromised may help to ensure reliable sensing of ventricular events and reliable detection of arrhythmias.

Each of the alternate sensing vectors stored in memory may have an assigned ranking value that defines which of the alternate sensing vectors the IMD selects after determining that the sensing integrity of the primary sensing vector is compromised. As described herein, a first alternate sensing vector of the plurality of alternate sensing vectors may be the sensing vector that the IMD selects upon a determination that the integrity of the primary sensing vector is compromised. A second alternate sensing vector of the plurality of alternate sensing vectors may be the next sensing vector that the IMD may switch to as the primary sensing vector in the case that the newly selected primary sensing vector (i.e., the first alternate sensing vector) becomes compromised.

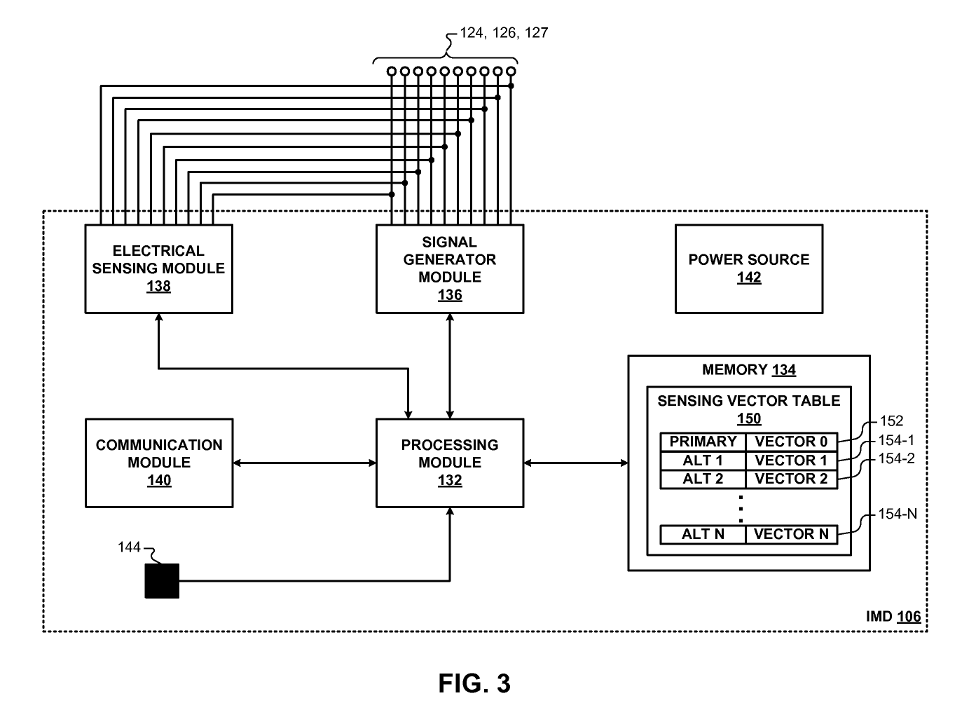

Selection of alternate sensing vectors for use in place of a current primary sensing vector is illustrated and described herein with reference to a sensing vector table. The sensing vector table may be a representation of the sensing vectors stored in memory, and may provide a graphical depiction of the procedure the IMD may use when selecting an alternate sensing vector for use as a new primary sensing vector. Example sensing vector tables (e.g., 150, 156, 158) are illustrated in FIGS. 3, 4A-4B, 7A-7C, and 9A-9B. As described herein, the sensing vector table may include the primary sensing vector and the alternate sensing vectors. The primary sensing vector, illustrated at the top of the sensing vector tables 150, 156, 158, is the sensing vector that the IMD may use for sensing ventricular events and for detecting arrhythmias (e.g., using a rate based detection algorithm). Below the primary sensing vector is the plurality of alternate sensing vectors. As described above, the IMD may select one of the alternate sensing vectors and set the selected alternate sensing vector as the new primary sensing vector upon a determination that the sensing integrity of the primary sensing vector is compromised.

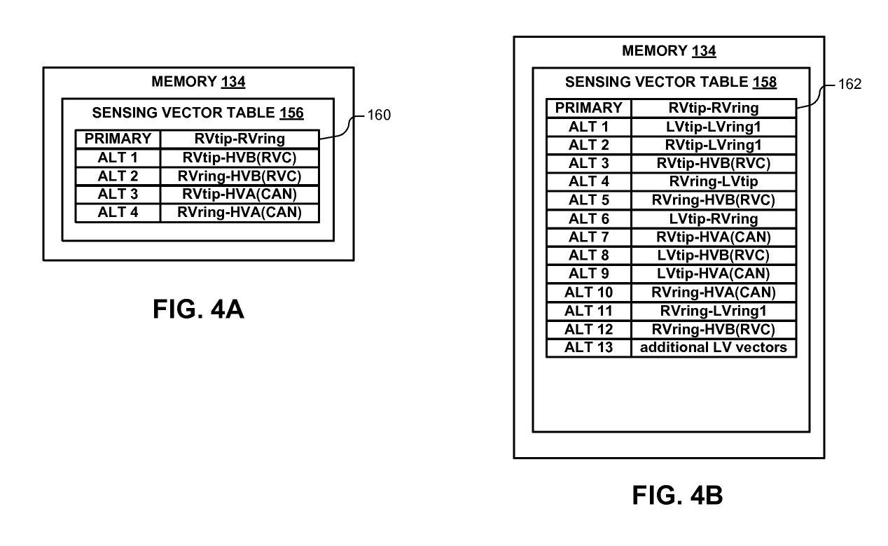

The IMD of the present disclosure may include a plurality of alternate sensing vectors. For example, as described with respect to FIGS. 4A-4B, the IMD may include 4-13 alternate sensing vectors. The number of alternate sensing vectors included in memory may depend on the number of different electrodes from which the sensing vectors may be selected. For example, an IMD having a greater number of electrodes may provide a greater number of alternate sensing vectors to choose from. Although 4-13 alternate sensing vectors are illustrated in FIGS. 4A-4B, it is contemplated that other numbers of alternate sensing vectors may be included in memory of an IMD.

The IMD may rank the alternate sensing vectors in order to construct an alternate sensing vector hierarchy that the IMD may select from when the IMD determines that the sensing integrity of the primary sensing vector has been compromised. The alternate sensing vector hierarchy is illustrated by the sensing vector tables (e.g., tables 150, 156, 158). The IMD may select the alternate sensing vector at the top of the hierarchy (e.g., at the top of the sensing vector table) in the event that the sensing integrity of the primary sensing vector is compromised.

The IMD may order the hierarchy of alternate sensing vectors based on the relative sensing integrity of the alternate sensing vectors. In other words, the IMD may form the hierarchy of alternate sensing vectors based on the integrity of the signals that may be acquired from the alternate sensing vectors. For example, the IMD may place an alternate sensing vector that is associated with a high signal integrity (e.g., higher signal integrity than other alternate sensing vectors) higher up on the hierarchy of the alternate sensing vectors. The IMD may place an alternate sensing vector that is associated with a low signal integrity (e.g., lower signal integrity than other alternate sensing vectors) toward the bottom of the hierarchy of the alternate sensing vectors.

As described herein, the IMD may select one of the alternate sensing vectors near the top of the hierarchy to use as a new primary sensing vector in the event that the sensing integrity of the primary sensing vector is compromised. In other words, the IMD may replace the primary sensing vector using an alternate sensing vector that may provide one of the highest sensing integrities relative to the other alternate sensing vectors. In some examples, the IMD may select the alternate sensing vector having the highest sensing integrity of the plurality of sensing vectors. Selecting an alternate sensing vector having a high sensing integrity to replace the primary sensing vector may provide for more reliable ventricular sensing upon switching to the new sensing vector.

Each of the alternate sensing vectors may be associated with a ranking value that indicates the sensing integrity of the alternate sensing vectors. In other words, the ranking value associated with an alternate sensing vector may indicate the integrity of a signal that may be acquired via that alternate sensing vector. The IMD may use the ranking values to determine the relative sensing integrity of each of the alternate sensing vectors. In other words, the IMD may determine, based on the ranking values, which of the alternate sensing vectors may provide the highest sensing integrity from the selection of possible alternate sensing vectors.

The ranking values associated with each of the alternate sensing vectors may indicate a relative rank (i.e., position) of the alternate sensing vectors to one another in terms of the sensing integrity of the alternate sensing vectors. In one example, the ranking values may be integer values that indicate the rank of the alternate sensing vectors relative to one another. For example, in the case where the memory includes five alternate sensing vectors, the five alternate sensing vectors may be assigned integer values of one to five. In this example, a ranking value of “1” may indicate the sensing vector having the highest sensing integrity, while the integer value “5” may indicate the sensing vector having the lowest sensing integrity.

Although the ranking values may be illustrated and described herein as consecutive integers that indicate the sensing integrity of different sensing vectors relative to one another, in some examples, the ranking values may include other values, such as nonconsecutive integers, decimal values, etc. In these examples, the magnitude of the ranking values may indicate the relative rankings, e.g., the largest values indicating an alternate sensing vector having the highest sensing integrity among the alternate sensing vectors.

The initial ranking values associated with the alternate vectors may be selected by a clinician or may take on default values (e.g. factory settings). In examples where the clinician programs the hierarchy of alternate sensing vectors, the IMD may assign consecutive integer values to the alternate sensing vectors based on the order selected by the clinician, with the first alternate sensing vector assigned a ranking value of “1”, and the Nth alternate sensing vector assigned a value of “N.” Example ranking values that may be initially programmed into the IMD are shown in the sensing vector table of FIG. 3, for example. In the example of FIG. 3, the first alternate sensing vector (i.e., ranking value 1) is the sensing vector “VECTOR 1”, the second alternate sensing vector (i.e., ranking value 2) is the sensing vector “VECTOR 2”, while the Nth alternate sensing vector (i.e., ranking value N) is the sensing vector “VECTOR N.”

The IMD may update the ranking values associated with the alternate sensing vectors during operation of the IMD while the IMD is implanted in the patient. The IMD may perform sensing integrity measurements on each of the sensing vectors (primary and alternates) in order to update the ranking values associated with the alternate sensing vectors. The IMD may perform a variety of different types of sensing integrity measurements in order to assign ranking values to the alternate sensing vectors and in order to determine when to set one of the alternate sensing vectors as the primary sensing vector. Example integrity measurements that may be performed on the sensing vectors may include, but are not limited to, impedance measurements, noise measurements, and signal amplitude measurements. Additional integrity measurements may include waveform morphology measurements, signal to noise ratio measurements, and other signal measurements, such as slew rate, signal frequency content, signal amplitude variability, and signal level and variability during cardiac diastole. Some of these sensing integrity measurements are now described in turn.

The IMD may perform impedance measurements of each of the sensing vectors in order to determine an impedance associated with the primary and alternate sensing vectors. A high impedance or a fluctuating impedance associated with a sensing vector may indicate a lead/electrode fracture or an issue with the electrical connection of a lead to the housing of the IMD. A consistently low or intermittently low impedance may indicate an insulation issue with the lead that may be resulting in an electrical short between electrodes. The IMD may assign a low ranking value, indicative of low sensing integrity, to an alternate sensing vector when a high, low or varying impedance is associated with a sensing vector. A low ranking value assigned to an alternate sensing vector may tend to push the alternate sensing vector towards the bottom of the alternate sensing vector hierarchy, which may help ensure that the IMD does not select the alternate sensing vector as a replacement when the sensing integrity of the primary sensing vector is compromised. In examples where the IMD detects a high impedance or a varying impedance in the primary sensing vector, the IMD may determine that the sensing integrity of the primary sensing vector is compromised.

The IMD may perform noise measurements in order to determine an amount of noise included in the signals sensed from different sensing vectors. Noise may be induced by EMI, interactions between leads and the IMD, and interactions between the leads and tissue (e.g., a tricuspid valve), for example. The IMD may assign lower ranking values to alternate sensing vectors that pick up a greater amount of noise during sensing, since noise present in an acquired signal may be indicative of a reduction in sensing integrity. A low ranking value assigned to an alternate sensing vector may tend to push the alternate sensing vector towards the bottom of the alternate sensing vector hierarchy, which may help ensure that the IMD does not select a sensing vector having a greater amount of noise than other alternate sensing vectors when the sensing integrity of the primary sensing vector is compromised. In examples where the IMD detects noise in the primary sensing vector, the IMD may determine that the sensing integrity of the primary sensing vector is compromised.

The IMD may perform signal amplitude measurements on the sensing vectors in order to determine a magnitude of the cardiac electrical signals that may be detected using different sensing vectors. The IMD may assign a lower ranking value to alternate sensing vectors that tend to acquire lower amplitude signals since analysis of low amplitude signals (e.g., <3 mV for R-waves) and detection of ventricular events in the low amplitude signals may prove more difficult and less reliable than analysis and detection of ventricular events in larger amplitude signals. In other words, cardiac signals acquired on a sensing vector that have larger amplitudes may indicate that the sensing vector has a higher sensing integrity, while cardiac signals acquired from a sensing vector having a low amplitudes may indicate that the sensing vector has a lower sensing integrity. The lower ranking value assigned to alternate sensing vectors that acquire low amplitude signals may help to ensure that the IMD does not set the primary sensing vector to an alternate sensing vector that acquires relatively low amplitude signals. In examples where the IMD determines that the amplitude of the cardiac electrical signals acquired via the primary sensing vector is relatively low (e.g., less than 3 mV), the IMD may determine that the sensing integrity of the primary sensing vector is compromised.

Although the IMD may perform the example integrity measurements described above (e.g., impedance, noise, signal amplitude) in order to determine the sensing integrity of an alternate sensing vector, in some examples, the IMD may perform different tests in order to determine the sensing integrity of a sensing vector. For example, the IMD may perform self-tests on the detection circuitry, signal to noise ratio tests, morphology tests, pacing capture detection tests, and/or specific lead fault detection routines involving injection of a known signal into the leads to determine whether the sensing circuit accurately detects that injected signal.

In some examples, the IMD may use a single one of the sensing integrity measurements to determine the relative sensing integrities of the alternate sensing vectors. For example, the IMD may rank the alternate sensing vectors based on a signal amplitude measured using the sensing vectors. In this example, the IMD may assign the highest ranking value to the alternate sensing vector having the highest signal amplitude, and may assign the lowest ranking value to the alternate sensing vector having the lowest signal amplitude. In another example where the IMD may use a single one of the sensing integrity measurements to determine the relative sensing integrities of the alternate sensing vectors, the IMD may rank the alternate sensing vectors based on the amount of noise associated with the alternate sensing vectors. In this example, the IMD may assign the highest ranking value to the alternate sensing vector having the least amount of noise, and may assign the lowest ranking value to the sensing vector having the greatest amount of noise. Although the IMD may rank alternate sensing vectors based on a single one of the sensing integrity measurements in some examples, in other examples the IMD may assign ranking values to the alternate sensing vectors based on multiple different sensing integrity measurements performed on each of the alternate sensing vectors.

The IMD may periodically update the ranking values associated with the alternate sensing vectors so that the alternate sensing vector table may be kept current in case a change from the primary sensing vector to an alternate sensing vector is desirable. In some examples, the IMD may update the ranking values immediately upon detection of issues with the primary sensing vector so that if an issue occurs with the primary sensing vector, the alternate sensing vector table may immediately provide a currently reliable sensing vector as a replacement to the primary sensing vector.

The primary and alternate sensing vectors may be initially programmed into the memory of the IMD prior to implantation in the patient, or upon implantation into the patient, e.g., by a clinician or by factory default settings. The order of the alternate sensing vectors may be selected initially based on the assumption that the alternate sensing vectors do not include potential sensing integrity issues. In other words, the alternate sensing vectors may be programmed into the IMD in an order that may not be based on potential faults in the IMD (e.g., potential lead fractures) or other sources of noise that may be present while the device is implanted in the patient.

In some examples, the IMD of the present disclosure may determine the sensing integrity of the primary sensing vector based on an accuracy with which the IMD detects arrhythmias using the primary sensing vector. The IMD may detect arrhythmias (e.g., VT/VF) based on a heart-rate detected using the primary sensing vector. Subsequent to detection of an arrhythmia, the IMD may use one or more algorithms in order to confirm or negate the existence of the detected arrhythmia. In some examples, the IMD of the present disclosure may determine the sensing integrity of the primary sensing vector based on a number of confirmations and negations of detected arrhythmias. Generally, the IMD may determine that the primary sensing vector has a higher sensing integrity when the arrhythmias detected using the primary sensing vector are confirmed. The IMD may determine that the primary sensing vector has a lower sensing integrity, e.g., may be compromised, when arrhythmias detected using the primary sensing vector are not confirmed, but instead, determined to be inappropriately detected. Determination of the sensing integrity of primary sensing vector based on confirmations and negations of detected arrhythmias is described hereinafter with respect to detection of shockable arrhythmias using the primary sensing vector.

The IMD of the present disclosure may provisionally detect shockable arrhythmias (e.g., VT/VF) based on a heart rate, heart rate onset, heart rate stability, electrogram morphology, etc. of the patient detected using the primary sensing vector. The IMD may be programmed to deliver high-energy therapy in response to detection of a shockable arrhythmia in order to correct the arrhythmia and return the patient's heart rate to a normal rhythm. However, in some examples, the IMD may perform secondary checks in order to confirm or negate the existence of the arrhythmia as provisionally detected based on sensed events that were sensed using the primary sensing vector.

Subsequent to detection of a shockable arrhythmia, the IMD may perform a secondary check on the cardiac electrical signal that led to the detection of the shockable arrhythmia in order to confirm or negate the presence of the arrhythmia before delivering therapy. In some examples, the IMD may determine, using the secondary check, that detection of the shockable arrhythmia was inappropriate. In other words, the IMD may determine, using the secondary check, that the IMD made an error when detecting the shockable arrhythmia using the primary sensing vector. In response to a determination that the shockable arrhythmia was detected in error, the IMD may withhold the delivery of high-energy therapy to the patient. Withholding of therapy in response to a determination that a shockable arrhythmia was wrongly detected may be an indicator that the sensing integrity of the primary sensing vector is compromised. Accordingly, in some examples, the IMD may determine that the sensing integrity of the primary sensing vectors is compromised based on a number of therapy withholdings.

The IMD of the present disclosure may count the number of times that the IMD withholds therapy after detection of arrhythmias via the primary sensing vector. The IMD may determine whether the sensing integrity of the primary sensing vector is compromised based on the number of times therapy is withheld. In some examples, the IMD may determine that the sensing integrity of the primary sensing vector is compromised when the number of withheld therapies is greater than a threshold number. In other examples, the IMD may determine that the sensing integrity of the primary sensing vector is compromised based on the number of withheld therapies relative to a total amount of detected shockable arrhythmias. For example, the IMD may determine that the sensing integrity of the primary sensing vector is compromised when the ratio of withheld therapies to the total number of detected shockable arrhythmias is greater than a threshold ratio.

The IMD may make the decision to withhold therapy using a variety of different algorithms. In some examples, the IMD may use a vector comparison algorithm in order to determine whether to withhold therapy. In this example, the IMD may compare the cardiac electrical data acquired using the primary sensing vector to other cardiac electrical data acquired using a different sensing vector (e.g., a far-field sensing vector). If the electrical data from the other sensing vector does not confirm the findings of the primary sensing vector, the IMD may determine that the arrhythmia detected using the primary sensing vector was detected in error. The IMD may withhold therapy based on the determination that the arrhythmia was detected in error. In other examples, the IMD may make the decision to withhold therapy based on findings using a template matching algorithm. For example, if the findings of the template matching algorithm do not confirm the findings of the primary sensing vector, the IMD may determine that the arrhythmia was detected in error and decide to withhold therapy.

In some examples, the IMD may use sensor data to either confirm or negate the detection of a shockable arrhythmia detected using the primary sensing vector. In these examples, the IMD may compare the cardiac electrical data acquired using the primary sensing vector to sensor data from a hemodynamic pressure sensor that indicates a physiological state (e.g., hemodynamic pressure) of the patient. If the hemodynamic sensor data does not confirm the findings of the primary sensing vector, the IMD may determine that the arrhythmia detected using the primary sensing vector was in error and the IMD may withhold therapy. In other examples, the IMD may compare the cardiac electrical data to other sensor data such as data acquired from an accelerometer or a heart sound sensor in order to confirm or negate the findings of the primary sensing vector. In still other examples, the IMD may determine when to withhold therapy based on assessment of environmental factors such as detected EMI noise, detection of 50/60Hz noise, detection of other noise, or based on patient interaction to inhibit therapy.

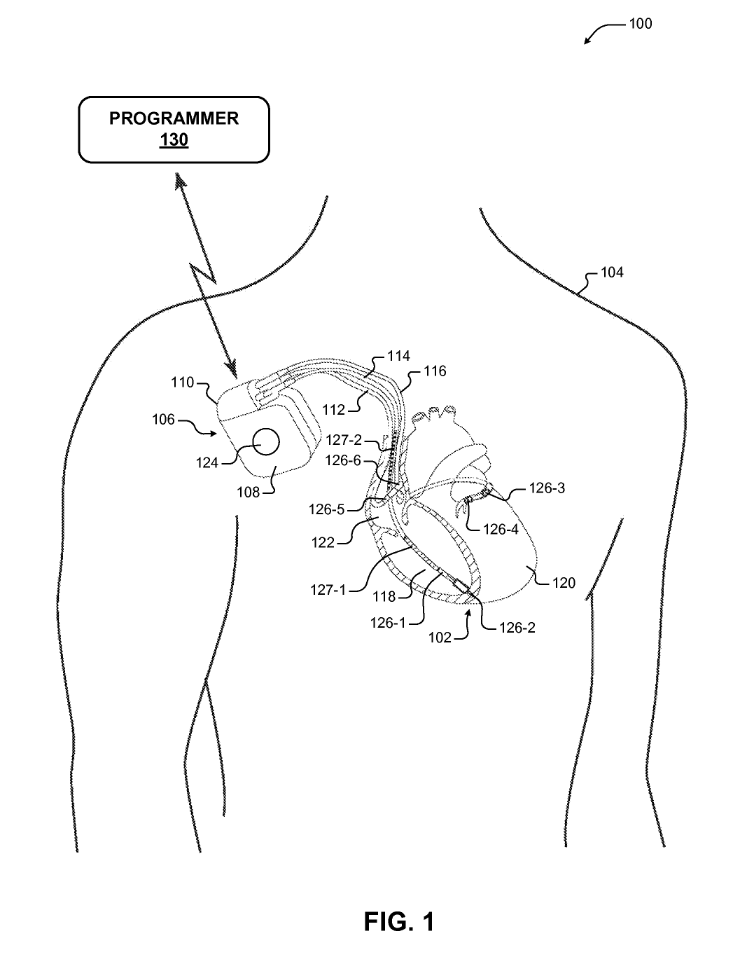

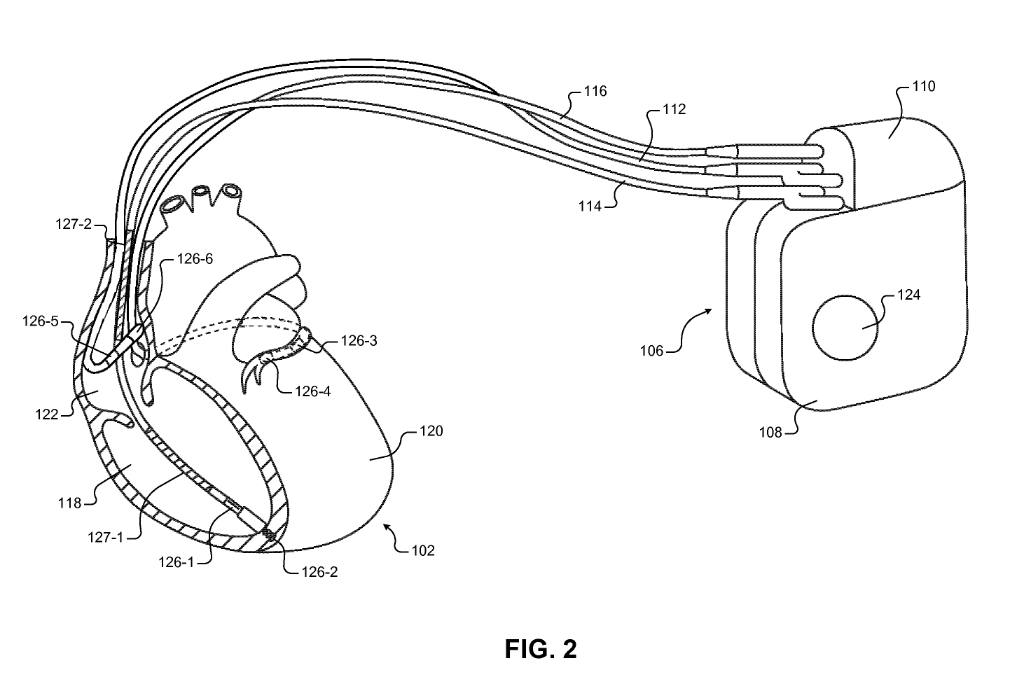

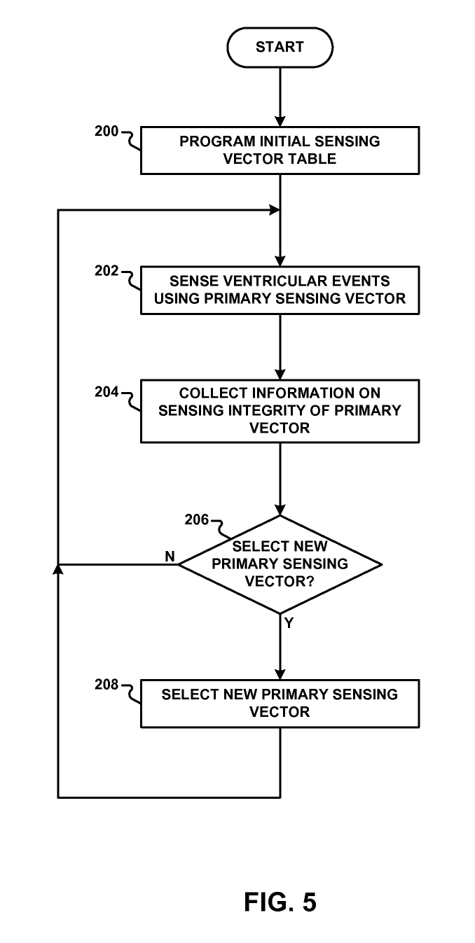

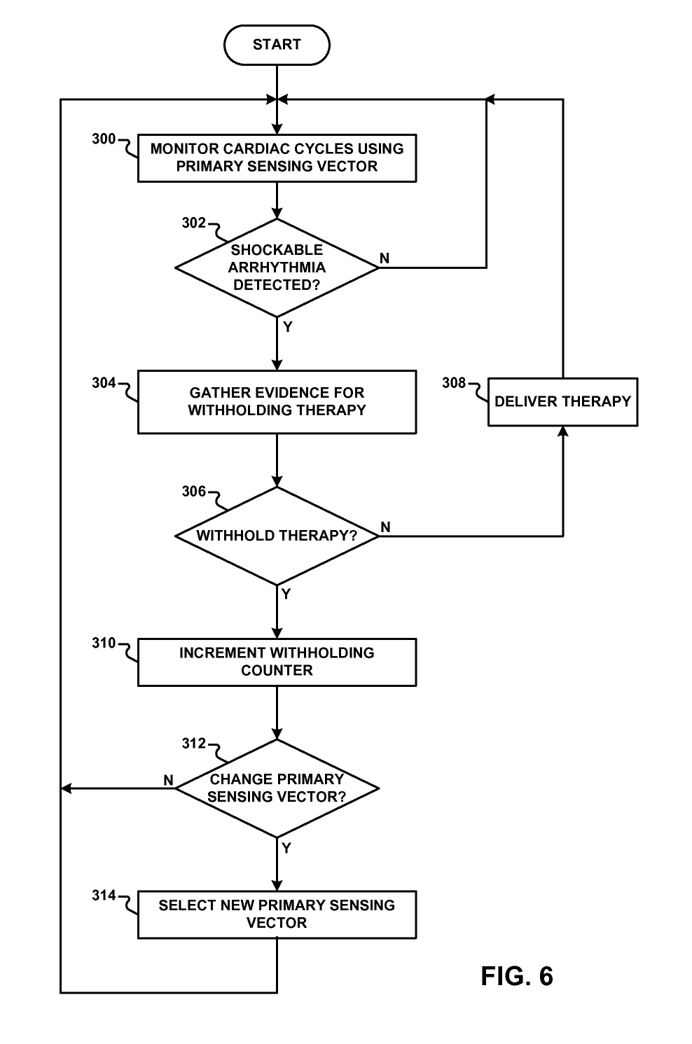

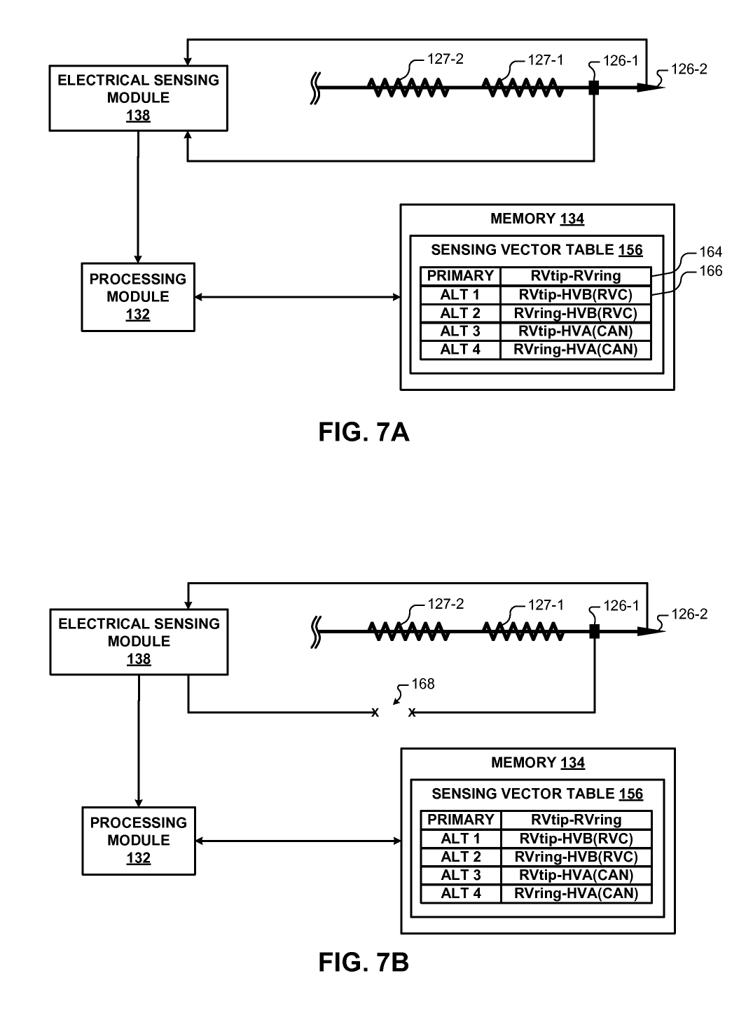

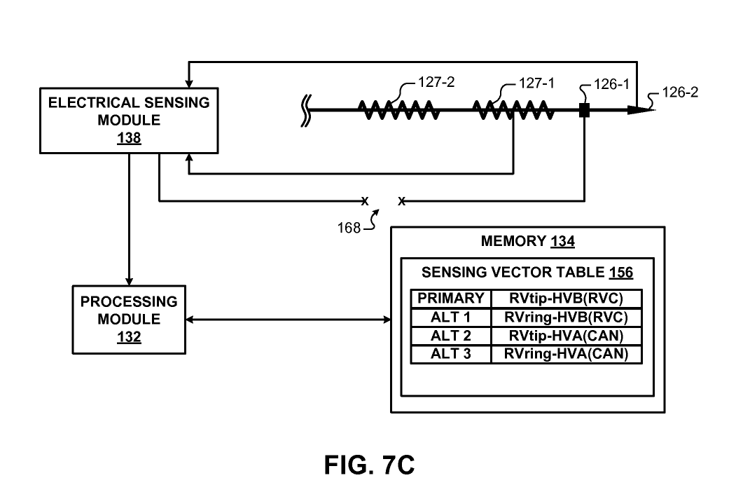

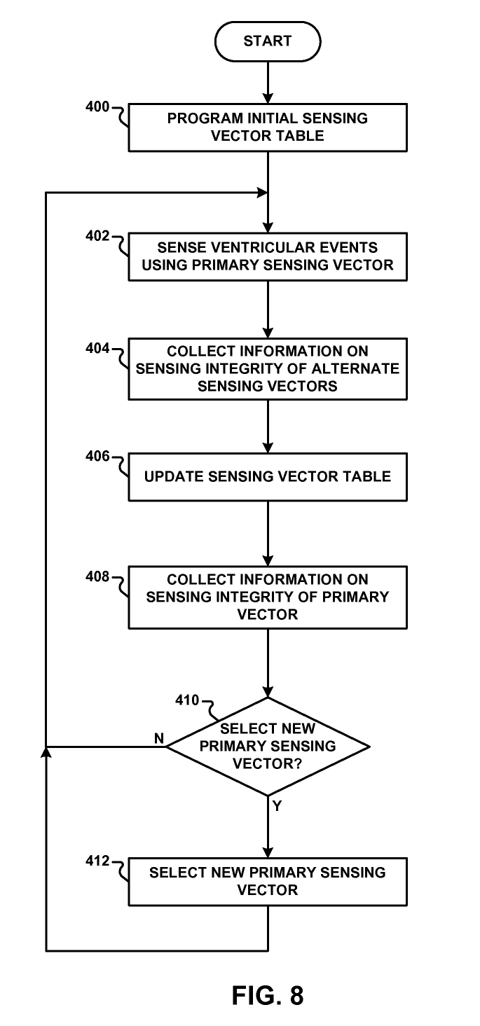

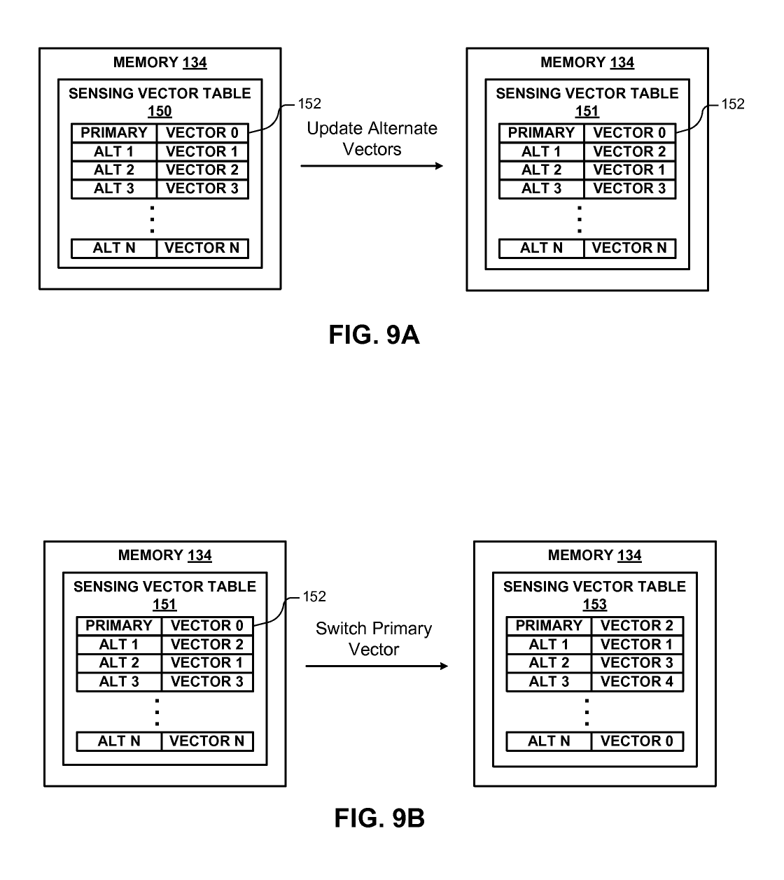

FIGS. 1-2 show an example system including an IMD that may sense ventricular events using a primary sensing vector, determine the sensing integrities of the primary sensing vector and alternate sensing vectors, and set one of the alternate sensing vectors as the primary sensing vector when the sensing integrity of the primary sensing vector is compromised. FIG. 3 shows an example functional block diagram of the IMD of FIGS. 1-2 including a memory that stores the primary and alternate sensing vectors. FIGS. 4A-4B show example sensing vector tables that illustrate example hierarchies of alternate sensing vectors. FIGS. 5-6 illustrate methods for setting an alternate sensing vector as the primary sensing vector. FIGS. 7A-7C are functional block diagrams that illustrate reconfiguration of the primary sensing vector in response to a determination that the sensing integrity of the primary sensing vector is compromised. FIG. 8 illustrates a method for updating the alternate sensing vectors. FIGS. 9A-9B illustrate example updates made to the alternate sensing vectors along with a subsequent reconfiguration of the primary sensing vector.

FIG. 1 shows an example system 100 that may be used to diagnose conditions of and provide therapy to a heart 102 of a patient 104. System 100 includes an IMD 106. For example, IMD 106 may be an implantable pacemaker, cardioverter, and/or defibrillator that monitors electrical activity of heart 102 and provides electrical stimulation to heart 102.

IMD 106 includes a housing 108 and a connector block 110. Housing 108 and connector block 110 may form a hermetic seal that protects components of IMD 106. IMD 106 is coupled to leads 112, 114, and 116 via connector block 110. Leads 112, 114, 116 extend into heart 102. Right ventricular lead 114 extends into right ventricle 118. Left ventricular coronary sinus lead 116 extends into the coronary sinus to a region adjacent to the free wall of left ventricle 120. Right atrial lead 112 extends into right atrium 122.

Housing 108 may enclose an electrical sensing module that monitors electrical activity of heart 102, and may also enclose a signal generator module that generates therapeutic stimulation, such as cardiac pacing pulses, ATP therapy, cardioversion therapy, and/or defibrillation therapy. Leads 112, 114, 116 are coupled to the signal generator module and the electrical sensing module of IMD 106 via connector block 110.

FIG. 2 shows a more detailed view of IMD 106 and leads 112, 114, 116. IMD 106 includes a housing electrode 124, which may be referred to as HVA electrode 124 or can electrode 124, which may be formed integrally with an outer surface of housing 108 of IMD 106 or otherwise coupled to housing 108. Although a single housing electrode 124 is illustrated in FIGS. 1-2, IMD 106 may include more or less than a single housing electrode 124.

Leads 112, 114, 116 include electrodes 126-1 to 126-6 (collectively “electrodes 126”). Lead 114 includes bipolar electrodes RVring 126-1 and RVtip 126-2 which are located in right ventricle 118. Lead 116 includes bipolar electrodes LVring1 126-3 and LVtip 126-4 which are located in the coronary sinus. Lead 112 includes bipolar electrodes 126-5, 126-6 which are located in right atrium 122. Electrodes 126-1, 126-3, 126-5 may take the form of ring electrodes. Electrodes 126-2, 126-4, 126-6 may take the form of, for example, helix tip electrodes or small circular electrodes at the tip of a tined lead or other fixation element. Lead 114 includes elongated electrodes 127-1, 127-2 (collectively “electrodes 127”) which may be coil electrodes. Electrode 127-1 may be referred to as HVB electrode 127-1 or as a right ventricular coil (RVC) electrode, and electrode 127-2 may be referred to as HVX electrode 127-2 or as a superior vena cava (SVC) coil electrode. Although three leads 112, 114, 116 are illustrated, systems according to the present disclosure may be implemented using more or less than 3 leads. Additionally, systems according to the present disclosure may be implemented using additional or fewer electrodes than illustrated in FIGS. 1-2.

Electrodes that may be used in sensing vectors may include, but are not limited to, electrodes on a right ventricular lead (e.g., RVtip 126-2, RVring 126-1, right ventricular coil HVB 127-1, and electrode HVX 127-2), electrodes on a left ventricular lead (e.g., LVtip 126-4 and LVring1 126-3, and additional LV ring electrodes in some examples), and the can electrode HVA 124. Ventricular sensing vectors may include electrodes on the same lead (e.g., RVtip-RVring, RVtip-HVB, RVtip-HVX, and LVtip-LVring1), electrodes on different leads (e.g., RVtip-LVring1), or an electrode on a lead in addition to the can electrode (e.g., RVtip-HVA).

IMD 106 may sense electrical activity of heart 102 and/or deliver electrical stimulation to heart 102 via electrodes 124, 126, 127. IMD 106 may sense electrical activity using any combination of electrodes 124, 126, 127. For example, IMD 106 may sense electrical activity via any bipolar combination of electrodes 126, 127. Furthermore, any of electrodes 126, 127 may be used for unipolar sensing in combination with housing electrode 124. IMD 106 may deliver pacing pulses using a unipolar or bipolar combination of electrodes 124, 126, 127. IMD 106 may deliver high-energy therapy (e.g., cardioversion pulses and/or defibrillation pulses) to heart 102 via any combination of elongated electrodes HVB 127-1, HVX 127-2, and housing electrode HVA 124.

Using the signal generator module and the electrical sensing module, IMD 106 may provide pacing pulses to heart 102 based on the electrical signals sensed within heart 102. IMD 106 may also provide ATP therapy, cardioversion, and/or defibrillation therapy to heart 102 based on the electrical signals sensed within heart 102. For example, IMD 106 may detect an arrhythmia of heart 102, such as VT/VF, and deliver ATP therapy, cardioversion, or defibrillation therapy to heart 102 in response to the detection of VT/VF.

Referring back to FIG. 1, system 100 may include a programmer 130. Programmer 130 may be a handheld computing device, desktop computing device, a networked computing device, etc. Programmer 130 may include a computer-readable storage medium having instructions that cause a processor of programmer 130 to provide the functions attributed to programmer 130 in the present disclosure. Programmer 130 may include a telemetry head (not shown). IMD 106 and programmer 130 may wirelessly communicate with one another, e.g., transfer data between one another, via the telemetry head. For example, IMD 106 may send data to programmer 130, and programmer 130 may retrieve data stored in IMD 106 and/or program IMD 106.

Data retrieved from IMD 106 using programmer 130 may include cardiac EGMs stored by IMD 106 that indicate electrical activity of heart 102 and marker channel data that indicates the occurrence and timing of sensing, diagnosis, and therapy events associated with IMD 106. Additionally, data may include information regarding the performance or integrity of IMD 106 or other components of diagnostic system 100, such as leads 112, 114, 116. Additionally, data may include information related to the sensing vectors, such as the ranking values associated with the alternate sensing vectors, and which sensing vectors, if any, are compromised. Data transferred to IMD 106 using programmer 130 may include, for example, values for operational parameters, information related to the sensing vectors, such as the initial order of the sensing vector tables, threshold values for measurements, such as an impedance threshold, amplitude thresholds, and noise thresholds. In some examples, data transferred to IMD 106 may include therapy withholding thresholds used to determine when the primary sensing vector is compromised.

FIG. 3 shows a functional block diagram of an example IMD 106. IMD 106 includes a processing module 132, memory 134, a signal generator module 136, an electrical sensing module 138, a communication module 140, and a power source 142, such as a battery, e.g., a rechargeable or non-rechargeable battery. In some examples, IMD 106 may include one or more sensors (e.g., sensor 144) with which processing module 132 may communicate. For example, sensor 144 may comprise at least one of a motion sensor (e.g., an accelerometer or piezoelectric element), a hemodynamic pressure sensor, and a heart sound sensor. Processing module 132 may determine, for example, an activity level of patient 104, a hemodynamic pressure of patient 104, and a heart rate of patient 104 based on data measured by sensor 144.

Modules included in IMD 106 represent functionality that may be included in IMD 106 of the present disclosure. Modules of the present disclosure may include any discrete and/or integrated electronic circuit components that implement analog and/or digital circuits capable of producing the functions attributed to the modules herein. For example, the modules may include analog circuits, e.g., amplification circuits, filtering circuits, and/or other signal conditioning circuits. The modules may also include digital circuits, e.g., combinational or sequential logic circuits, memory devices, etc. Memory may include any volatile, non-volatile, magnetic, or electrical media, such as a random access memory (RAM), read-only memory (ROM), non-volatile RAM (NVRAM), electrically-erasable programmable ROM (EEPROM), Flash memory, or any other memory device. Furthermore, memory may include instructions that, when executed by one or more processing circuits, cause the modules to perform various functions attributed to the modules herein.

The functions attributed to the modules herein may be embodied as one or more processors, hardware, firmware, software, or any combination thereof. Depiction of different features as modules is intended to highlight different functional aspects and does not necessarily imply that such modules must be realized by separate hardware or software components. Rather, functionality associated with one or more modules may be performed by separate hardware or software components, or integrated within common or separate hardware or software components.

Processing module 132 may communicate with memory 134. Memory 134 may include computer-readable instructions that, when executed by processing module 132, cause processing module 132 to perform the various functions attributed to processing module 132 herein. Memory 134 may include any volatile, non-volatile, magnetic, or electrical media, such as RAM, ROM, NVRAM, EEPROM, Flash memory, or any other digital media.

Processing module 132 may communicate with signal generator module 136 and electrical sensing module 138. Signal generator module 136 and electrical sensing module 138 are electrically coupled to electrodes 126, 127 of leads 112, 114, 116 and housing electrode 124. Electrical sensing module 138 is configured to monitor signals from electrodes 124, 126, 127 in order to monitor electrical activity of heart 102. Electrical sensing module 138 may selectively monitor any bipolar or unipolar combination of electrodes 124, 126, 127.

Signal generator module 136 may generate and deliver electrical stimulation therapy to heart 102 via electrodes 124, 126, 127. Electrical stimulation therapy may include at least one of pacing pulses, ATP therapy, cardioversion therapy, and defibrillation therapy. Processing module 132 may control signal generator module 136 to deliver electrical stimulation therapy to heart 102 according to one or more therapy programs, which may be stored in memory 134. For example, processing module 132 may control signal generator module 136 to deliver pacing pulses to heart 102 based on one or more therapy programs and signals received from electrical sensing module 138. In other examples, processing module 132 may control signal generator module 136 to deliver at least one of ATP therapy, cardioversion therapy, and defibrillation therapy when processing module 132 detects a tachyarrhythmia. For example, in the event that processing module 132 detects a tachyarrhythmia, processing module 132 may load an ATP regimen from memory 134, and control signal generator module 136 to implement the ATP regimen. In other examples, processing module 132 may implement a cardioversion regimen or a defibrillation regimen upon detection of a tachyarrhythmia.

Communication module 140 includes any suitable hardware, firmware, software or any combination thereof for communicating with another device, such as programmer 130 and/or a patient monitor. Under the control of processing module 132, communication module 140 may receive downlink telemetry from and send uplink telemetry to programmer 130 and/or a patient monitor with the aid of an antenna (not shown) in IMD 106.

Processing module 132 may instruct electrical sensing module 138 to acquire cardiac electrical signals using a primary sensing vector specified in memory 134. In response to the instruction from processing module 132, electrical sensing module 138 may acquire cardiac electrical signals using the indicated primary sensing vector. For example, electrical sensing module 138 may include analog circuits that acquire the cardiac electrical signals using the primary sensing vector, filter and amplify the cardiac electrical signals, and convert the analog cardiac electrical signals to digital values. Processing module 132 may receive the digitized data (i.e., raw data) of cardiac electrical activity generated by electrical sensing module 138.

Processing module 132 may sense ventricular events based on the data received from electrical sensing module 138. Processing module 132 may implement rate-based detection and analysis algorithms in order to detect and analyze arrhythmias based on sensed ventricular events. For example, processing module 132 may monitor the length of intervals between sensed ventricular events, and detect arrhythmias (e.g., VT/VF) when a predetermined number of those intervals are shorter than a programmed time interval. In some examples, processing module 132 may perform further analysis of arrhythmias using rate information. For example, processing module 132 may characterize arrhythmias based on the range of values in which the intervals fall, the stability of the intervals, and the average or median values of the intervals. In some examples, processing module 132 may also implement a template matching algorithm in order to determine the morphology of a detected arrhythmia and to further classify the arrhythmia.

Upon detection of potentially life-threatening arrhythmias (e.g., VT/VF), processing module 132 may instruct signal generator module 138 to treat the potentially life-threatening arrhythmia using high-energy therapies (e.g., cardioversion or defibrillation therapy). Potentially life-threatening arrhythmias (e.g., VT/VF) that are typically treated using high-energy therapies (e.g., cardioversion or defibrillation) may be referred to herein as “shockable arrhythmias.” Accordingly, in the event that processing module 132 detects a shockable arrhythmia, processing module 132 may instruct signal generator module 136 to deliver high-energy therapy to treat the shockable arrhythmia. Delivery of high-energy therapy by signal generator module 136 to heart 102 may correct the shockable arrhythmia and return heart 102 to a normal rhythm. In examples where the detected shockable arrhythmia is not corrected, processing module 132 may control delivery of subsequent high-energy therapies.

Memory 134 includes a sensing vector table 150 that includes a plurality of different ventricular sensing vectors. For example, sensing vector table 150 includes primary sensing vector 152 and alternate sensing vectors 154-1, 154-2, . . . , and 154-N (collectively “alternate sensing vectors 154”). Primary sensing vector 152 may be the electrode combination that processing module 132 uses to sense ventricular events (e.g., ventricular depolarizations). For example, processing module 132 may instruct electrical sensing module 138 to sense ventricular events using the electrode combination specified by primary sensing vector 152 in memory 134. Accordingly, processing module 132 may detect arrhythmias based on the ventricular events that are sensed using primary sensing vector 152. In the example of FIG. 3, primary sensing vector 152 is labeled as “VECTOR 0.” The phrase “VECTOR 0” in sensing vector table 150 may indicate an electrode combination. For example, the phrase “VECTOR 0” may indicate the electrode combination RVtip-RVring, or another electrode combination.

Typically, processing module 132 may sense ventricular events and detect arrhythmias using primary sensing vector 152 specified in memory 134. However, when processing module 132 determines that the integrity of primary sensing vector 152 is compromised (e.g., based on a detected fault or detected noise), processing module 132 may select one of alternate sensing vectors 154 from memory 134 and set the selected alternate sensing vector as primary sensing vector 152. Subsequently, processing module 132 may instruct electrical sensing module 138 to use the newly selected primary sensing vector to acquire cardiac electrical signals so that processing module 132 may sense ventricular events and detect arrhythmias using the newly selected primary sensing vector.

Alternate sensing vectors 154 may have an assigned ranking value that defines which of alternate sensing vectors 154 processing module 132 selects after detecting sensing integrity issues with primary sensing vector 152. As illustrated herein, the ranking value of alternate sensing vectors 154 is indicated using an integer value. In the example of FIG. 3, “ALT 1” indicates that the sensing vector “VECTOR 1” has a ranking value of “1.” Similarly, “ALT 2” and “ALT N” indicate that sensing vectors “VECTOR 2” and “VECTOR N” have ranking values of “2” and “N,” respectively.

In the example sensing vector table 150 of the present disclosure, alternate sensing vector “ALT 1” may be the sensing vector having the highest sensing integrity, as determined by processing module 132. Alternate sensing vectors further down sensing vector table 150, e.g., “ALT 2” to “ALT N,” may be sensing vectors having lower sensing integrity, as determined by processing module 132. In other words, a higher integer value associated with an alternate sensing vectors (i.e., a sensing vector on sensing table further from primary sensing vector 152) may indicate a relatively lower integrity sensing vector. Alternate sensing vectors “ALT 1” and “ALT 2” may be referred to herein as first and second alternate sensing vector 154-1, 154-2.

Although the ranking values may be illustrated and described herein as consecutive integers that indicate the sensing integrity of different sensing vectors relative to one another, in some examples, the ranking values may include other values, such as nonconsecutive integers, decimal values, etc. In these examples, the magnitude of the ranking values may indicate the relative rankings, e.g., the largest values indicating an alternate sensing vector having the highest sensing integrity among the alternate sensing vectors. In some examples, processing module 132 may determine whether to switch to an alternate sensing vector based on the magnitude of the ranking value associated with the alternate sensing vector. For example, if the magnitude of an alternate sensing vector indicates that the integrity of the sensing vector is relatively high (e.g., greater than a threshold magnitude), then processing module 132 may switch to the alternate sensing vector. Whereas, if the magnitude of an alternate sensing vector indicates that the integrity of the sensing vector is relatively low (e.g., less than the threshold magnitude) then processing module 132 may not switch to the alternate sensing vector. Such a threshold magnitude may be implemented by processing module 132 in order to help ensure that a switch to an alternate sensing vector is likely to result in a high quality alternative vector for sensing.

FIGS. 4A-4B show example sensing vector tables 156, 158 that may be included in memory 134. Sensing vector table 156 may be a sensing vector table included in an implantable cardioverter-defibrillator (ICD) having a single high-voltage coil located in the right ventricle. Sensing vector table 158 may be a sensing vector table included in an ICD having both a right ventricular lead and a left ventricular lead which include electrodes for sensing cardiac electrical activity of both the left and right ventricles, respectively.

The example sensing vector tables 156, 158 of FIGS. 4A-4B may be initially programmed by a clinician. For example, primary sensing vectors 160, 162 may initially be selected (e.g., by a clinician) based on the assumption that the sensing integrity of primary sensing vectors 160, 162 is not compromised. The order of the alternate sensing vectors of sensing vector tables 156, 158 may be selected initially (e.g., by the clinician) based on the assumption that the alternate sensing vectors do not include potential sensing integrity issues.

As illustrated and described with respect to FIGS. 4A-4B, the number of available alternate sensing vectors may depend on the number of electrodes that are available for sensing ventricular events. IMDs having a greater number of electrodes that are capable of sensing ventricular events may provide for a greater number of alternate sensing vectors in a sensing vector table. For example, sensing vector table 158 of FIG. 4B may include a greater number of alternate sensing vectors than sensing vector table 156 because sensing vector table 158 is included in an IMD having sensing electrodes on a left ventricular lead (e.g., LVtip and LVring1). It is contemplated that other sensing vector tables, other than those shown in FIGS. 4A-4B, may be programmed into memory 134 of IMD 106. For example other sensing vector tables may include a greater number or a lesser number of alternate sensing vectors than the number of sensing vectors illustrated in FIGS. 4A-4B. Additionally, other sensing vector tables may include different electrode combinations than those illustrated in FIGS. 4A-4B.

Referring back to FIG. 3, processing module 132 may select one of alternate sensing vectors 154 from memory 134 when processing module 132 determines that the integrity of primary sensing vector 152 has been compromised. Processing module 132 may then set the selected alternate sensing vector as primary sensing vector 152. In some examples, processing module 132 may set first alternate sensing vector 154-1 as primary sensing vector 152 when processing module 132 determines that the sensing integrity of primary sensing vector 152 is compromised. For example, if processing module 132 determines that the sensing integrity of “VECTOR 0” is compromised, processing module 132 may set primary sensing vector 152 to first alternate sensing vector 154-1 “VECTOR 1.” With respect to FIG. 4A, assuming sensing vector table 156 is included in memory 134 of IMD 106, processing module 132 may set first alternate sensing vector RVtip-HVB(RVC) as the primary sensing vector, in place of RVtip-RVring, when processing module 132 determines that the integrity of the primary sensing vector, RVtip-RVring, is compromised. Reconfiguration of primary sensing vector 152 is described in further detail hereinafter with respect to FIGS. 5-9.

FIG. 5 shows a method for reconfiguring a primary sensing vector in response to a determination that the sensing integrity of the primary sensing vector is compromised. Initially, memory 134 may be programmed with an initial sensing vector table 150 (200). Example sensing vector tables (e.g., 150, 156, 158) are illustrated in FIGS. 3-4. Processing module 132 may sense ventricular events during operation of IMD 106 using primary sensing vector 152 of sensing vector table 150 (202).

Processing module 132 may collect information related to the sensing integrity of primary sensing vector 152 during operation of IMD 106 (204). For example, processing module 132 may perform a variety of different types of sensing integrity measurements in order to collect information related to the sensing integrity of primary sensing vector 152. Example integrity measurements that may be performed on the sensing vectors may include, but are not limited to, impedance measurements, noise measurements, and signal amplitude measurements.

With respect to impedance measurements, processing module 132 may monitor the impedance of primary sensing vector 152 and may determine whether the sensing integrity of primary sensing vector 152 is compromised based on the monitored impedance. Processing module 132 may monitor the impedance of primary sensing vector 152 by instructing electrical sensing module 138 to perform impedance measurements on primary sensing vector 152. In some examples, processing module 132 may determine that the sensing integrity of primary sensing vector 152 is compromised when the measured impedance is greater than a threshold impedance that indicates a lead/electrode fracture or an issue with the electrical connection of a lead to the housing of the IMD.

With respect to noise measurements, processing module 132 may monitor an amount of noise included in signals received via primary sensing vector 152 and may determine whether the sensing integrity of primary sensing vector 152 is compromised based on the amount of noise included in the signal. In some examples, processing module 132 may determine that the sensing integrity of primary sensing vector 152 is compromised when the measured amount of noise is greater than a threshold amount of noise that may indicate that sensing ventricular events via primary sensing vector 152 is not sufficiently reliable.

With respect to signal amplitude measurements, processing module 132 may monitor the amplitude of signals obtained via primary sensing vector 152 and may determine whether the sensing integrity of primary sensing vector 152 is compromised based on the amplitude of the signals. In some examples, processing module 132 may determine that the sensing integrity of primary sensing vector 152 is compromised when the amplitude of signals received via primary sensing vector 152 is less than a threshold amplitude that may indicate that sensing ventricular events via primary sensing vector 152 is not sufficiently reliable.

Although processing module 132 may perform the example integrity measurements described above (e.g., impedance, noise, and signal amplitude) in order to determine whether primary sensing vector 152 is compromised, in some examples, processing module 132 may perform different tests in order to determine the sensing integrity of a sensing vector. In some examples, processing module 132 may use only a single one of the integrity measurements to determine whether the sensing integrity of primary sensing vector 152 is compromised. In other examples, processing module 132 may use multiple different sensing integrity measurements to determine whether the integrity of primary sensing vector 152 is compromised.

In some examples, processing module 132 may determine whether the sensing integrity of primary sensing vector 152 is compromised based on a number of times therapy is withheld from patient 104 after processing module 132 initially detects a shockable arrhythmia. During operation of IMD 106, processing module 132 may provisionally detect shockable arrhythmias based on a detected heart rate that is determined using primary sensing vector 152. Subsequent to a provisional detection of a shockable arrhythmia, processing module 132 may perform secondary checks in order to confirm or negate the existence of the detected shockable arrhythmia. Processing module 132 may withhold the delivery of high-energy therapy in response to a determination that the shockable arrhythmia was detected in error. Processing module 132 may determine whether the sensing integrity of the primary sensing vector is compromised based on the number of times processing module 132 has withheld therapy. A more detailed description of determining when to switch from primary sensing vector 152 to one of alternate sensing vectors 154 based on a number of withheld therapies is described with respect to the method of FIG. 6.

With respect to block (206) of FIG. 5, processing module 132 may determine whether to select a new primary sensing vector based on the information related to the sensing integrity of primary sensing vector 152 that was collected in block (204). Processing module 132 may select a new primary sensing vector in block (208) when processing module 132 determines that the sensing integrity of primary sensing vector 152 is compromised based on the information collected in block (204). Processing module 132 may continue sensing ventricular events using primary sensor 152 in block (202) when processing module 132 determines that the sensing integrity of primary sensing vector 152 is not compromised in block (206). In some examples, when processing module 132 selects a new primary sensing vector in block (208), processing module 132 may adjust detection algorithms to account for the change. For example, processing module 132 may reconfigure a t-wave oversensing algorithm or adjust an EMI detection algorithm for the new primary sensing vector, which may prevent any sensing configuration issues that may arise with the new primary sensing vector, e.g., unwanted far-field sensing of muscle activity.

Processing module 132 may determine that the sensing integrity of primary sensing vector 152 is compromised based on one or more of the sensing integrity measurements described above and/or based on a number of withheld therapies. In some examples, processing module 132 may determine that the sensing integrity of primary sensing vector 152 is compromised when one of the integrity sensing measurements indicates that primary sensing vector 152 is compromised. For example, processing module 132 may determine that the sensing integrity of primary sensing vector 152 is compromised when processing module 132 determines that an impedance associated with primary sensing vector 152 is greater than a threshold impedance. In another example, processing module 132 may determine that the sensing integrity of primary sensing vector 152 is compromised when processing module 132 determines that an amount of noise present in signals acquired using primary sensing vector 152 is greater than a threshold amount of noise. In another example, processing module 132 may determine that the sensing integrity of primary sensing vector 152 is compromised when processing module 132 determines that the amplitude of cardiac electrical signals acquired via primary sensing vector 152 is less than a threshold amplitude. In still other examples, processing module 132 may determine that the sensing integrity of primary sensing vector 152 is compromised when processing module 132 determines that a number of withheld therapies is greater than a threshold number of withheld therapies.

In some examples, processing module 132 may determine that the sensing integrity of primary sensing vector 152 is compromised based on a single type of measurement, e.g., based on one of the measured impedance associated with primary sensing vector 152, the amount of noise associated with primary sensing vector 152, the signal amplitude associated with primary sensing vector 152, or the number of withheld therapies.

In some examples, processing module 132 may require detection of a plurality of sensing issues with primary sensing vector 152 before processing module determines that the sensing integrity of primary sensing vector 152 is compromised. For example, processing module 132 may require that the impedance of primary sensing vector 152 be greater than the threshold impedance for a threshold number of impedance measurements (e.g., for a threshold amount of time) before processing module 132 determines that the sensing integrity of primary sensing vector 152 is compromised. In another example, processing module 132 may require that the amount of noise present in signals acquired via primary sensing vector 152 be greater than the threshold amount of noise for a threshold number of noise measurements (e.g., for a threshold amount of time) before processing module 132 determines that the sensing integrity of primary sensing vector 152 is compromised. In another example, processing module 132 may require that the signal amplitude of signals acquired via primary sensing vector 152 be less than the threshold signal amplitude for a threshold number of measurements (e.g., for a threshold amount of time) before processing module 132 determines that the sensing integrity of primary sensing vector 152 is compromised.

In some examples, processing module 132 may require that more than one of the sensing integrity measurements (e.g., impedance, noise, amplitude) indicate that the sensing integrity of primary sensing vector 152 is compromised before making a determination that primary sensing vector 152 is compromised in block (206). For example, processing module 132 may require that both the impedance of primary sensing vector 132 be greater than a threshold impedance and that the amount of noise detected on primary sensing vector 152 be greater than a threshold amount of noise before processing module 132 determines that the sensing integrity of primary sensing vector is compromised.

Processing module 132 may select a new primary sensing vector from alternate sensing vectors 154 in block (208) when processing module 132 determines that the sensing integrity of primary sensing vector 152 has been compromised. In some examples, processing module 132 may select first alternate sensing vector 154-1 from sensing vector table 150 when processing module 132 determines that the sensing integrity of primary sensing vector 152 has been compromised. In some examples, first alternate sensing vector 154-1 may be the first alternate sensing vector, as initially programmed in block (200), e.g., by a clinician, or by default. In other examples, as described herein with respect to FIGS. 8-9, processing module 132 may reorder alternate sensing vectors 154 during operation of IMD 106 in order to place the alternate sensing vector having the highest sensing integrity in the position of first alternate sensing vector 154-1. In these examples, processing module 132 may select a sensing vector in the position of first alternate sensing vector 154-1 that is different from the sensing vector that was initially programmed into the position of first alternate sensing vector 154-1 in block (200), e.g., when processing module 132 has reordered alternate sensing vectors 154.

FIG. 6 shows a method for reconfiguring a primary sensing vector in response to a determination that the sensing integrity of the primary sensing vector is compromised based on a number of withheld therapies. Initially, processing module 132 monitors cardiac cycles using the initially programmed primary sensing vector 152 (300). For example, processing module 132 may sense ventricular events using primary sensing vector 152 and detect arrhythmias based on the sensed ventricular events.

Based on the ventricular events sensed using primary sensing vector 152, processing module 132 may determine whether patient 104 is experiencing a shockable arrhythmia (302). If processing module 132 does not detect a shockable arrhythmia, processing module 132 may continue monitoring cardiac cycles using primary sensing vector 152 in block (300). If processing module 132 detects a shockable arrhythmia, processing module 132 may gather evidence for making a determination of whether to withhold therapy (304).

Processing module 132 may gather evidence for withholding therapy in a variety of ways. In some examples, processing module 132 may perform a secondary check on the cardiac electrical signal that led to the detection of the shockable arrhythmia in order to gather evidence for withholding therapy. In one example, processing module 132 may use a vector comparison algorithm in order to acquire evidence for determining whether to withhold therapy. In this example, processing module 132 may compare the cardiac electrical data acquired using primary sensing vector 152 to other cardiac electrical data acquired using a different sensing vector (e.g., a far field sensing vector including HVA 124). If the electrical data from the other sensing vector also indicates the presence of a shockable arrhythmia, as detected by processing module 132 using primary sensing vector 152, then this finding may indicate that therapy should be delivered to heart 102. In other words, this finding may not provide evidence that therapy should be withheld.

However, if processing module 132 determines that the electrical data from other sensing vectors do not indicate a shockable arrhythmia, as detected using primary sensing vector 132, then this finding may provide evidence that the sensing integrity of primary sensing vector 152 is compromised. Processing module 132 may decide to withhold high-energy therapy to heart 102 based on the determination that the electrical data from other sensing vectors do not indicate a shockable arrhythmia, as detected using primary sensing vector 152.

In some examples, processing module 132 may use data acquired from sensor 144 that may indicate a physiological state of patient 104 in order to gather evidence for withholding therapy in block (304). For example, processing module 132 may compare the cardiac electrical data acquired using primary sensing vector 152 to data acquired by sensor 144 in order to gather evidence for withholding therapy. If the sensor data indicating a physiological state of patient 104 also indicates the presence of a shockable arrhythmia, as detected by processing module 132 using primary sensing vector 154, then this finding may indicate that therapy should be delivered to heart 102. In other words, this finding using sensor data may not provide evidence that therapy should be withheld.

However, if processing module 132 determines that the sensor data does not indicate a shockable arrhythmia, as detected using primary sensing vector 152, then this finding may provide evidence that the sensing integrity of primary sensing vector 152 is compromised. Processing module 132 may decide to withhold high-energy therapy to heart 102 based on the determination that the sensor data does not indicate a shockable arrhythmia, as detected using primary sensing vector 152.

In one example, sensor 144 may include a hemodynamic pressure sensor that generates data indicating a hemodynamic pressure, e.g., in right ventricle 118 or in the pulmonary artery. Processing module 132 may detect a shockable arrhythmia (e.g., VT/VF) based on the frequency components present in the signal received from sensor 144 and/or based on a drop in pressure indicated by sensor 144 when sensor 144 includes a hemodynamic pressure sensor. In another example, sensor 144 may include a blood oxygen sensor. Processing module 132 may detect a shockable arrhythmia (e.g., VT/VF) based on fluctuations/drops in the oxygen concentration as indicated by sensor 144.

Although processing module 132 may gather evidence for withholding therapy by performing secondary checks on primary sensing vector 154 using other sensing vectors and a variety of sensor data, in other examples, processing module 132 may gather evidence for withholding therapy using other techniques, such as template matching algorithms, patient feedback/inhibition of therapy, patient activity/respiration, sensing, self-test of sensing circuit integrity, focused tests on lead pathway integrity, etc.

Processing module 132 may determine whether to withhold therapy based on the gathered evidence (306). If the secondary checks (e.g., other sensing data and sensor data) indicate the presence of a shockable arrhythmia, then processing module 132 may control signal generator module 136 to deliver high-energy therapy (308). However, if the secondary checks do not indicate the presence of a shockable arrhythmia, as detected based on ventricular events sensed using primary sensing vector 152, processing module 132 may withhold therapy for the detected shockable arrhythmia.

Processing module 132 may include a therapy withholding counter that processing module 132 may increment in order to keep track of a total number of times therapy is withheld from heart 102. Processing module 132 may increment the withholding counter (310) when processing module 132 decides to withhold therapy in block (306). Generally, a greater number of withheld therapies may more reliably indicate that the sensing integrity of primary sensing vector 152 is compromised, while a lesser number of withheld therapies may more reliably indicate that the sensing integrity of primary sensing vector 152 is not compromised. Accordingly, a larger withholding counter may indicate more reliably that the sensing integrity of primary sensing vector 152 is compromised, while a smaller withholding counter may indicate more reliably that the sensing integrity of primary sensing vector 152 is not compromised.

Processing module 132 may include a withholding counter threshold. Processing module 132 may determine whether to change primary sensing vector 152 based on the magnitude of the withholding counter relative to the withholding counter threshold (312). The withholding counter threshold may be selected such that a withholding counter value that is greater than the withholding counter threshold indicates that the sensing integrity of primary sensing vector 152 is compromised, while a withholding counter value that is less than the withholding counter threshold may indicate that the sensing integrity of primary sensing vector 152 is not compromised.

Processing module 132 may continue monitoring cardiac cycles using the same primary sensing vector in block (300) when the withholding counter value is less than the withholding counter threshold. Processing module 132 may select a new primary sensing vector when the withholding counter value is greater than the withholding counter threshold (314). For example, processing module 132 may select one of alternate sensing vectors 154 (e.g., first alternate sensing vector 154-1) and set the one of alternate sensing vectors 154 as the new primary sensing vector in block (314). As described above, in some examples, processing module 132 may determine whether to switch to alternate sensing vector 154-1 also based on the magnitude (e.g., decimal value) of the ranking value associated with alternate sensing vector 154-1 relative to a threshold magnitude.

FIGS. 7A-7C are functional block diagrams that illustrate detection of a mechanical fault in primary sensing vector 152 and subsequent reconfiguration of primary sensing vector 152. With respect to FIG. 7A, processing module 132 may instruct electrical sensing module 138 to sense ventricular events using sensing vector RVtip-RVring which includes electrodes RVtip 126-2 and RVring 126-1. First alternate sensing vector 164 is set as RVtip-HVB(RVC) and second alternate sensing vector 166 is set as RVring-HVB(RVC).

FIG. 7B illustrates a break 168 in the conductor that connects electrode RVring 126-1 to electrical sensing module 138. Processing module 132 may detect break 168 in the conductor that connects RVring 126-1 to electrical sensing module 138 using a lead impedance test. For example, processing module 132 may detect a high impedance between electrodes RVtip 126-2 and RVring 126-1 that is indicative of a lead fracture between RVtip 126-2 and RVring 126-1. Processing module 132 may determine that the sensing integrity of sensing vector RVtip-RVring is compromised based on the high impedance detected between the electrodes RVtip 126-2 and RVring 126-1. Accordingly, processing module 132 may reconfigure primary sensing vector RVtip-RVring and update the alternate sensing vectors. For example, processing module 132 may set the first alternate sensing vector RVtip-HVB(RVC) as the new primary sensing vector.

FIG. 7C illustrates ventricular sensing after processing module 132 has updated sensing vector table 156 by setting sensing vector RVtip-HVB(RVC) (i.e., the prior first alternate sensing vector) as the primary sensing vector. As illustrated in FIG. 7C, the conductor connecting electrode HVB(RVC) 127-1 does not include a mechanical fault, such as a break, that may cause a high impedance in sensing vector RVtip-HVB(RVC). Accordingly, the reconfiguration of the primary sensing vector proved successful in overcoming the mechanical fault (i.e., break 168) that compromised the sensing integrity of the prior primary sensing vector RVtip-RVring.

FIG. 7C also illustrates an example method for updating alternate sensing vectors in sensing vector table 156. In FIG. 7C processing module 132 deleted the prior primary sensing vector RVtip-RVring as a selectable sensing vector in response to the determination that the sensing integrity of sensing vector RVtip-RVring was compromised. Additionally, processing module 132 shifted each of the alternate sensing vectors up one rank when processing module 132 set the first alternate sensing vector RVtip-HVB(RVC) of FIG. 7B as the new primary sensing vector. If processing module 132 determines in the future that the sensing integrity of the primary sensing vector RVtip-HVB(RVC) is compromised, processing module 132 may set the first alternate sensing vector of FIG. 7C (i.e., RVring-HVB(RVC)) as the new primary sensing vector and delete the primary sensing vector RVtip-HVB(RVC) from sensing vector table 156.

FIG. 8 is a flowchart that illustrates an example method for updating alternate sensing vectors 154 of sensing vector table 150. Initially, memory 134 may be programmed with an initial sensing vector table 150 (400). Example sensing vector tables (e.g., 150, 156, 158) are illustrated in FIGS. 3-4. Processing module 132 may sense ventricular events during operation of IMD 106 using primary sensing vector 152 of sensing vector table 150 (402).

Processing module 132 may then collect information related to the sensing integrity of alternate sensing vectors 154 during operation of IMD 106 (404). For example, processing module 132 may perform a variety of different types of sensing integrity measurements in order to collect information related to the sensing integrity of alternate sensing vectors 154. Example sensing integrity measurements performed on alternate sensing vectors 154 may be similar to those sensing integrity measurements performed in block (204) of FIG. 5 with respect to primary sensing vector 152. For example, processing module 132 may perform at least one of impedance measurements, noise measurements, and signal amplitude measurements on alternate sensing vectors 154 to determine a sensing integrity associated with alternate sensing vectors 154.

In some examples, processing module 132 may perform sensing integrity measurements on each of alternate sensing vectors 154 to determine a sensing integrity associated with each of alternate sensing vectors 154. Processing module 132 may assign a ranking value to each of alternate sensing vectors 154 (i.e., a rank) based on the outcome of the sensing integrity measurements. In some examples, processing module 132 may perform a single type of sensing integrity measurement (e.g., an impedance measurement) on each of alternate sensing vectors 154 and subsequently assign ranking values to each of alternate sensing vectors 154 based on the single type of sensing integrity measurement. In other examples, processing module 132 may perform multiple types of sensing integrity measurements (e.g., impedance, noise, and amplitude) on each of alternate sensing vectors 154 and subsequently assign ranking values to each of alternate sensing vectors 154 based on the multiple sensing integrity measurements.

Although processing module 132 may perform sensing integrity measurements on each of alternate sensing vectors 154 to determine a sensing integrity associated with each of alternate sensing vectors 154, in other examples, processing module 132 may perform sensing integrity measurements on only a portion of alternate sensing vectors 154, e.g., the top two or three alternate sensing vectors. Performing sensing integrity measurements on only a portion of alternate sensing vectors 154 may reduce an amount of time and power expended by IMD 106 when performing sensing integrity measurements for the purpose of reordering alternate sensing vectors 154.

Processing module 132 may update the ranking values of alternate sensing vectors 154 in sensing vector table 150 (406) based on the outcome of the sensing integrity measurements on alternate sensing vectors 154 in block (404). In examples where processing module 132 ranks alternate sensing vectors 154 based on a single type of sensing integrity measurement (e.g., impedance), processing module 132 may rank alternate sensing vectors 154 based on the outcome of that single type of measurement. For example, processing module 132 may assign higher ranking values to alternate sensing vectors having associated impedance values that are not high enough to indicate that the sensing integrity of those vectors is compromised. Alternatively, processing module 132 may assign lower ranking values to alternate sensing vectors that have high impedance values indicative of lead fracture. In this manner, processing module 132 may update the hierarchy of alternate sensing vectors 154 such that alternate sensing vectors having impedance values indicative of mechanical failure are ranked toward the bottom of sensing vector table 150. These alternate sensing vectors ranked toward the bottom of sensing vector table 150 are less likely to be selected by processing module 132 in the case that the sensing integrity of primary sensing vector 152 is compromised. In other words, in a scenario where processing module 132 determines that the sensing integrity of primary sensing vector 152 is compromised, processing module 132 may be more likely to reconfigure the primary sensing vector 152 to a new sensing vector that is less likely to present a high impedance to electrical sensing module 138.

In some examples, processing module 132 may assign ranking values to alternate sensing vectors 154 based on an amount of noise present in signals acquired from alternate sensing vectors 154. For example, processing module 132 may assign higher ranking values to alternate sensing vectors that pick up less noise than those alternate sensing vectors that pick up a greater amount of noise. In this manner, processing module 132 may update the hierarchy of alternate sensing vectors 154 such that alternate sensing vectors associated with a greater amount of noise are ranked toward the bottom of sensing vector table 150. These alternate sensing vectors ranked toward the bottom of sensing vector table 150 are less likely to be selected by processing module 132 in the case that the sensing integrity of primary sensing vector 152 is compromised. In other words, in a scenario where processing module 132 determines that the sensing integrity of primary sensing vector 152 is compromised, processing module 132 may be more likely to reconfigure primary sensing vector 152 to a new sensing vector that is less likely to be corrupted with noise.

In some examples, processing module 132 may assign ranking values to alternate sensing vectors 154 based on the magnitude of the cardiac electrical signals acquired from alternate sensing vectors 154. For example, processing module 132 may assign higher ranking values to alternate sensing vectors that acquire cardiac electrical signals having greater amplitude than alternate sensing vectors that acquire cardiac electrical signals having a smaller amplitude. In this manner, processing module 132 may update the hierarchy of alternate sensing vectors 154 such that alternate sensing vectors that acquire cardiac electrical signals that are smaller in amplitude are ranked toward the bottom of sensing vector table 150. These alternate sensing vectors ranked toward the bottom of sensing vector table 150 are less likely to be selected by processing module 132 in the case that the sensing integrity of primary sensing vector 152 is compromised. In other words, in a scenario where processing module 132 determines that the sensing integrity of primary sensing vector 152 is compromised, processing module 132 may be more likely to reconfigure primary sensing vector 152 to a new sensing vector that is less likely to acquire cardiac electrical signals having small amplitudes.

Although processing module 132 may rank alternate sensing vectors 154 based on a single sensing integrity measurement (e.g., impedance, noise, or amplitude), in some examples, processing module 132 may rank alternate sensing vectors 154 based on multiple sensing integrity measurements. For example, processing module 132 may assign a ranking value to each of alternate sensing vectors 154 based on a sensing integrity of each of the alternate sensing vectors determined based on multiple sensing integrity measurements, e.g., at least two of impedance measurements, noise measurements, and amplitude measurements.

FIG. 9A shows an example how processing module 132 may update sensing vector table 150. Sensing vector table 150 on the left in FIG. 9A may represent an initial sensing vector table programmed into memory 134 (e.g., by a clinician). Sensing vector table 151 on the right may represent a sensing vector table that has been updated to reflect newly determined sensing integrities associated with each of the alternate sensing vectors included in sensing vector tables 150, 151. In the example of FIG. 9A, processing module 132 may have determined, after performing one or more sensing integrity measurements on the first alternate sensing vector of table 150, that sensing vector “VECTOR 2” had a sensing integrity that was relatively greater than the sensing integrity associated with alternate sensing vector “VECTOR 1.” Accordingly, processing module 132 assigned sensing vector “VECTOR 2” a higher ranking value than sensing vector “VECTOR 1.” Similarly, processing module 132 may have determined that sensing vector “VECTOR 1” had a sensing integrity that was relatively greater than the sensing integrity associated with sensing vector “VECTOR 3.” Accordingly, processing module 132 assigned sensing vector “VECTOR 1” a higher ranking value than sensing vector “VECTOR 3.”

Referring back to FIG. 8, in block (408), processing module 132 may collect information related to the sensing integrity of primary sensing vector 152 during operation of IMD 106. For example, processing module 132 may perform a variety of different types of sensing integrity measurements in order to collect information related to the sensing integrity of primary sensing vector 152. Example integrity measurements that may be performed on the primary sensing vector 152 may include, but are not limited to, impedance measurements, noise measurements, and signal amplitude measurements.

Processing module 132 may then determine whether to select a new primary sensing vector (410) based on the information related to the sensing integrity of primary sensing vector 152 that was collected in block (408). In a similar manner as that described with respect to block (206) of FIG. 5, processing module 132 may select a new primary sensing vector in block (412) when processing module 132 determines that the sensing integrity of primary sensing vector 152 is compromised based on the information collected in block (408). Processing module 132 may continue sensing ventricular events using primary sensor 152 in block (402) when processing module 132 determines that the sensing integrity of primary sensing vector 152 is not compromised in block (410).

FIG. 9B shows the selection of a new primary sensing vector in the case that processing module 132 determines that the sensing integrity of primary sensing vector 152 of FIG. 9A is compromised. In the example of FIG. 9B, processing module 132 set the first alternate sensing vector “VECTOR 2” of sensing vector table 151 on the left of FIG. 9B as the new primary sensing vector in response to a determination that the sensing integrity of primary sensing vector 152 on the left of FIG. 9B was compromised. Processing module 132 set primary sensing vector “VECTOR 0” to the Nth rank in sensing vector table 153 in response to a determination that the sensing integrity of primary sensing vector “VECTOR 0” had been compromised. Processing module 132 also shifted each of the alternate sensing vectors up one rank after setting the first alternate sensing vector “VECTOR 2” as the new primary sensing vector. In this manner, sensing vector “VECTOR 0,” which was determined to have a compromised sensing integrity, is placed at the bottom of sensing vector table 153 so that sensing vector “VECTOR 0” is not chosen as the primary sensing vector in the future in response to a determination that the sensing integrity of the new primary sensing vector “VECTOR 2” is compromised.

Although the above description and figures are directed to sensing ventricular events using a ventricular sensing vector and subsequent reconfiguration of the ventricular sensing vector, the systems and methods of the present disclosure may be applicable to reconfiguring pacing vectors of the IMD that may be used to pace the atria or ventricles. A “pacing vector” may generally refer to a ventricular pacing vector or an atrial pacing vector. A ventricular pacing vector may be a pair of electrodes used to pace the ventricles. An atrial pacing vector may be a pair of electrodes used to pace the atria.

An IMD according to the present disclosure may provide cardiac pacing therapy to a patient using a current pacing vector (i.e., a primary pacing vector), monitor the integrity of one or more alternate pacing vectors, and switch from the current pacing vector to an alternate pacing vector when the integrity of the current pacing vector is compromised. In some examples, an IMD may be configured to switch from a current pacing vector to an alternate pacing vector, but may not be configured to switch from one sensing vector to another sensing vector in the manner described above. In other examples, an IMD may be configured to switch sensing vectors and switch pacing vectors. The criteria for switching a pacing vector may be similar to, or different from, the criteria used by the IMD to determine when to switch sensing vectors. Switching between different pacing vectors based on the integrity of the pacing vectors may help to ensure adequate pacing therapy (e.g., for bradycardia therapy) even when the integrity of some pacing vectors are compromised.

Automatic switching between pacing vectors may be accomplished in a manner that is similar to that described above with respect to switching between sensing vectors. For example, the IMD may determine when the primary pacing vector is compromised based on a detection of a fault associated with the primary pacing vector, detected noise associated with the primary pacing vector, or a detected decrease in the amplitude of signals acquired via electrodes associated with the primary pacing vector. In response to determining that the primary pacing vector is compromised, the IMD may select one of a plurality of alternate pacing vectors from memory and set the selected alternate pacing vector as the new primary pacing vector. Each of the alternate pacing vectors from which the IMD may select may be associated with a ranking value that indicates the integrity of that alternate pacing vector. The IMD may select the alternate pacing vector that corresponds to a ranking value indicating the highest integrity. Subsequently, the IMD may use the newly selected primary pacing vector to pace the atria and ventricles.

In some examples, the IMD of the present disclosure may update the ranking values of the alternate pacing vectors during operation. The IMD may perform a variety of different types of measurements in order to update the ranking values. Example measurements that may be performed by the IMD to determine the integrity of the alternate pacing vectors may include, but are not limited to, impedance measurements, noise measurements, capture threshold measurements, and signal amplitude measurements.

Various examples have been described. These and other examples are within the scope of the following claims.

What is claimed is:

1. A system comprising:
a memory comprising a primary sensing vector and N alternate sensing vectors, wherein N is an integer that is greater than 1; and
a processing module configured to:
determine a ranking value for each of the N alternate sensing vectors, wherein each ranking value is indicative of the integrity of a cardiac electrical signal acquired via the corresponding alternate sensing vector;
sense cardiac events using the primary sensing vector;
detect a reduction in the integrity of a cardiac electrical signal acquired via the primary sensing vector;
select one of the N alternate sensing vectors in response to detecting a reduction in the integrity of the cardiac electrical signal acquired via the primary sensing vector, the selection based on the ranking value associated with the one of the N alternate sensing vectors; and
sense cardiac events using the selected one of the N alternate sensing vectors.

2. The system of claim 1, wherein the processing module is configured to periodically update the ranking values for each of the N alternate sensing vectors.

3. The system of claim 1, wherein the processing module is configured to perform one or more integrity measurements on each of the N alternate sensing vectors, and wherein the processing module determines the ranking value for each of the N alternate sensing vectors based on the one or more integrity measurements.

4. The system of claim 3, wherein the one or more integrity measurements include at least one of an impedance measurement for determining an impedance of an electrical pathway, a noise measurement for determining an amount of noise in the cardiac electrical signal, and a signal amplitude measurement for determining an amplitude of the cardiac electrical signal.

5. The system of claim 1, wherein the magnitudes of the ranking values indicate the relative integrity of the cardiac electrical signals acquired via the alternate sensing vectors.

6. The system of claim 5, wherein the processing module selects the one of the N alternate sensing vectors by selecting the one of the N alternate sensing vectors having a ranking value that indicates the highest integrity amongst the N alternate sensing vectors.

7. The system of claim 1, wherein the processing module is configured to perform one or more integrity measurements on the primary sensing vector, and wherein the processing module is configured to detect the reduction in the integrity of the cardiac electrical signal acquired via the primary sensing vector based on the one or more integrity measurements.

8. The system of claim 7, wherein the one or more integrity measurements include measurements of at least one of an impedance of the primary sensing vector, an amount of noise included in the acquired cardiac electrical signal, and an amplitude of the acquired cardiac electrical signal.

9. The system of claim 8, wherein the processing module is configured to detect the reduction in the integrity of the cardiac electrical signal acquired via the primary sensing vector when the impedance of the primary sensing vector increases to a value that is greater than a threshold impedance, when an amount of noise included in the acquired cardiac electrical signal increases to a value that is greater than a threshold amount of noise, or when the amplitude of the acquired cardiac electrical signal decreases to a value that is less than a threshold amplitude.

10. The system of claim 1, wherein the cardiac events sensed using the primary sensing vector and the cardiac events sensed using the selected one of the N alternate sensing vectors are ventricular depolarizations.

11. The system of claim 1, wherein the cardiac events sensed using the primary sensing vector and the cardiac events sensed using the selected one of the N alternate sensing vectors are atrial depolarizations.

12. A method comprising:
storing a primary sensing vector and N alternate sensing vectors in a memory, wherein N is an integer that is greater than 1;
determining a ranking value for each of the N alternate sensing vectors, wherein each ranking value is indicative of the integrity of a cardiac electrical signal acquired via the corresponding alternate sensing vector;
sensing cardiac events using the primary sensing vector;
detecting a reduction in the integrity of a cardiac electrical signal acquired via the primary sensing vector;
selecting one of the N alternate sensing vectors in response to detecting a reduction in the integrity of the cardiac electrical signal acquired via the primary sensing vector, the selection based on the ranking value associated with the one of the N alternate sensing vectors; and
sensing cardiac events using the selected one of the N alternate sensing vectors.

13. The method of claim 12, further comprising periodically updating the ranking values for each of the N alternate sensing vectors.

14. The method of claim 12, further comprising:
performing one or more integrity measurements on each of the N alternate sensing vectors; and
determining the ranking value for each of the N alternate sensing vectors based on the one or more integrity measurements.

15. The method of claim 12, further comprising:
performing one or more integrity measurements on the primary sensing vector; and
detecting the reduction in the integrity of the cardiac electrical signal acquired via the primary sensing vector based on the one or more integrity measurements.

16. A method comprising:
sensing a plurality of ventricular events using a first ventricular sensing vector;
detecting a plurality of arrhythmias based on analysis of the plurality of sensed ventricular events;
determining whether to withhold therapy for each of the plurality of detected arrhythmias;
determining a number of times that therapy was withheld for the plurality of detected arrhythmias; and
determining when to switch from the first ventricular sensing vector to a second ventricular sensing vector based on the number of times therapy was withheld.

17. The method of claim 16, further comprising:
determining whether a sensing vector other than the first ventricular sensing vector indicates the presence of the plurality of detected arrhythmias; and
withholding therapy when the sensing vector other than the first ventricular sensing vector does not confirm the detection of an arrhythmia that was detected based on the ventricular events sensed using the first ventricular sensing vector.

18. The method of claim 16, further comprising:
comparing the number of times therapy was withheld to a threshold value; and
switching from the first ventricular sensing vector to the second ventricular sensing vector when the number of times therapy was withheld is greater than the threshold value.

19. The method of claim 16, further comprising:
determining a ratio of the number of times therapy was withheld to the number of arrhythmias detected;
comparing the ratio to a ratio threshold; and
switching from the first ventricular sensing vector to the second ventricular sensing vector when the ratio is greater than the ratio threshold.

20. A system comprising:
a memory comprising a first ventricular sensing vector and a second ventricular sensing vector; and
a processing module configured to:
sense a plurality of ventricular events using the first ventricular sensing vector;
detect a plurality of arrhythmias based on analysis of the plurality of sensed ventricular events;
determine whether to withhold therapy for each of the plurality of detected arrhythmias;
determine a number of times that therapy was withheld for the plurality of detected arrhythmias; and
determine when to switch from the first ventricular sensing vector to the second ventricular sensing vector based on the number of times therapy was withheld.

21. A system comprising:
a memory comprising a primary pacing vector and N alternate pacing vectors, wherein N is an integer that is greater than 1; and
a processing module configured to:
determine a ranking value for each of the N alternate pacing vectors, wherein each ranking value is indicative of the integrity of the corresponding alternate pacing vector;
pace one of the atria and the ventricles using the primary pacing vector;
detect a reduction in the integrity of the primary pacing vector;
select one of the N alternate pacing vectors in response to detecting a reduction in the integrity of the primary pacing vector, wherein the selection is based on the ranking value associated with the one of the N alternate pacing vectors; and
pace the one of the atria and the ventricles using the selected one of the N alternate pacing vectors.

22. The system of claim 21, wherein the processing module is configured to periodically update the ranking values for each of the N alternate pacing vectors.

23. The system of claim 21, wherein the processing module is configured to perform one or more integrity measurements on each of the N alternate pacing vectors, and wherein the processing module determines the ranking value for each of the N alternate pacing vectors based on the one or more integrity measurements.

24. The system of claim 23, wherein the one or more integrity measurements include at least one of an impedance measurement for determining an impedance of an electrical pathway, and a noise measurement for determining an amount of noise.

25. The system of claim 21, wherein the processing module is configured to perform one or more integrity measurements on the primary pacing vector, and wherein the processing module is configured to detect the reduction in the integrity of the primary pacing vector based on the one or more integrity measurements.
